(19) United States
(12) Patent Application Publication
(10) Pub. No.: US 2006/0233740 A1
(43) Pub. Date: Oct. 19, 2006

(54) Conjugates of an hGH moiety and a polymer

(76) Inventors: Mary J. Bossard, Madison, AL (US); Ping Zhang, Madison, AL (US)

Correspondence Address: NEKTAR THERAPEUTICS, 150 INDUSTRIAL ROAD, SAN CARLOS, CA, 94070, US

(21) Appl. No.: 11/388,784

(22) Filed: Mar. 23, 2006

Related U.S. Application Data
(60) Provisional application No. 60/664,401, filed on Mar. 23, 2005.

(51) Int. Cl. A61K 38/27; C08G 63/91; C08G 63/48

(52) U.S. Cl. 424/78.27; 514/12; 525/54.1

(57) ABSTRACT

Conjugates of an hGH moiety and one or more non-peptidic water-soluble polymers are provided. Typically, the non-peptidic water-soluble polymer is poly(ethylene glycol) or a derivative thereof. Also provided are compositions comprising such conjugates, methods of making the conjugates, and methods of administering compositions comprising such conjugates to a mammalian subject.

### CROSS REFERENCE TO RELATED APPLICATIONS

This application claims the benefit of priority to Provisional Patent Application No. 60/664,401, filed Mar. 23, 2005, the contents of which are hereby incorporated herein by reference.

### FIELD OF THE INVENTION

Among other things, one or more embodiments of the present invention relate generally to conjugates comprising an hGH moiety (i.e., a moiety having human growth hormone activity) and a polymer. In addition, the invention relates to (among other things) compositions comprising conjugates, methods for synthesizing conjugates, and methods for treating patients.

### BACKGROUND OF THE INVENTION

Growth Hormone (“GH,” also referred to as “somatotropin”) is a nonglycosylated protein having 191 amino acids with a molecular weight of about 22,000 Daltons. Two disulfide bonds connect the side chains of internal cysteine residues; there is one disulfide bond at positions 53 and 165, and one at positions 182 and 189. In healthy humans, GH is produced by the anterior lobe of the pituitary gland (or adenohypophysis) and produces both metabolic and anabolic effects. Metabolically, GH can produce an initial insulin-like effect with increased tissue uptake of glucose and amino acids with a concomitant decrease in lipolysis. Anabolically, GH can result in longitudinal growth, which is mediated by GH's ability to stimulate somatomedins (or insulin-like growth factors) that, in turn, promote the uptake of sulfate into cartilaginous tissues. Thus, in humans, growth hormone deficiency leads to inadequate somatomedin production and potentially dwarfism.

In children, criteria for the diagnosis of GH deficiency have been defined as a growth rate less than four centimeters per year and the absence of a serum GH response to two growth hormone secretagogues. Congenital growth hormone deficiency has a prevalence of about 1:4000. In children suffering from growth hormone deficiency, the administration of exogenous GH can result in normalized growth such that the predicted adult height for such children can be achieved.

Due to its relatively short half-file (endogenous GH has a half-life of about 20-25 minutes), the recommended frequency for dosing exogenous GH can be as often as daily. Since hGH therapy typically requires daily injections, patients, and in particular, pediatric patients, dislike the inconvenience and discomfort associated with this regimen.

One proposed solution to these problems has been to provide a sustained release composition containing human growth hormone (hGH). For example, U.S. Pat. No. 5,654,010 describes a composition comprising a polymeric matrix of a biocompatible polymer and particles of biologically active, stabilized hGH, wherein the particles are dispersed within the biocompatible polymer. Such compositions, however, may not provide sufficient activity over the desired period of time and may result in an undesirable initial “burst” effect or release of a relatively large amount of hGH in an uncontrolled manner.

Others have suggested the use of PEGylation technology, or the attachment of a poly(ethylene glycol) derivative to a growth hormone, in order to prolong hGH's in vivo half-life. For example, International Patent Publication WO 99/03887 describes poly(ethylene glycol)-hGH conjugates prepared by reacting hGH with specific poly(ethylene glycol) reagents [sometimes referred to as “activated poly(ethylene glycol)s]. The described poly(ethylene glycol) reagents result in specific conjugates having specific structures.

International Patent Publication WO 99/03887 also describes preparing a mutein of hGH having a cysteine residue introduced within the protein and then PEGylating the mutein at the introduced cysteine residue via a PEG-maleimide derivative.

U.S. Patent Application Publication Nos. 2003/0171285 and 2004/0038892 describe poly(ethylene glycol)-hGH conjugates prepared by a variety of activated poly(ethylene glycol)s. The structures of the corresponding conjugates are, of course, limited to the particular structures of the activated poly(ethylene glycol)s described in the publication.

U.S. Patent Application Publication No. 2004/0127417 describes linear and branched versions of activated poly(ethylene glycol)s containing an aldehyde, thereby relying on aldehyde chemistry to direct the selectivity of the poly(ethylene glycol) to the N-terminus. Example 1 of this publication provides an activated branched poly(ethylene glycol) having the following structure:

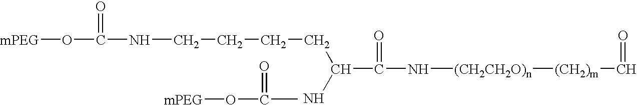

### Activated Branched Poly(ethylene glycol) Used in Example 1 of U.S. Patent Publication 2004/0127417

This activated poly(ethylene glycol) incorporates a lysine residue, as illustrated below: lysine residue 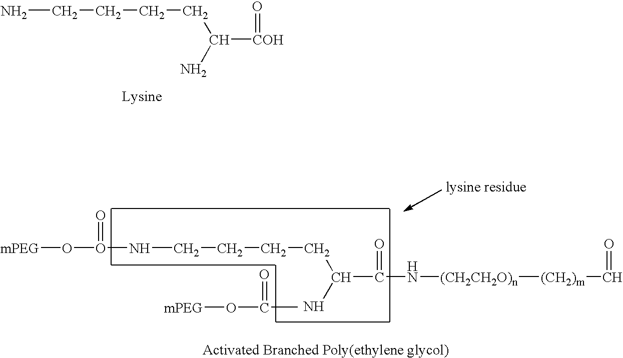
The structure of the corresponding hGH-polymer conjugate prepared using the above activated branched poly(ethylene glycol) would also incorporate the lysine residue.

Notwithstanding these described conjugates, however, it remains advantageous to provide conjugates of hGH capable of providing one or more advantages over previously-described compositions; conjugates formed from PEG derivatives having different weight average molecular weights (e.g., greater than about 10,000); and conjugates formed from recombinant hGH and not muteins or fusion proteins thereof.

Thus, there remains a need in the art to provide additional conjugates of water-soluble polymers and moieties having hGH activity, preferably having one or more advantageous features suitable for providing a superior hGH therapeutic composition. Ideally, a conjugate of the invention will possess one or more of the following features and/or advantages over current hGH formulations: can be readily synthesized in good yields; can be prepared with enhanced selectivity to favor a particular conjugate type (e.g., hGH having one water-soluble polymer covalently attached thereto, hGH having two water-soluble polymers covalently attached thereto, etc.); can be purified to provide a substantially homogeneous conjugate composition (e.g., a composition of substantially all monomers, i.e., having only one polymer covalently attached to hGH, a composition of substantially all dimers, i.e., having only two polymers covalently attached to hGH, and/or well-defined mixtures of particular conjugate species, etc.); and possess a degree of biologic activity in vivo (or in a suitable in vitro model), such that a balance between in vivo activity and pharmacokinetics and/or pharmacodynamics is achieved, to thereby provide a conjugate having properties superior to unmodified hGH and previously-described hGH conjugates. Among other things, one or more embodiments of the present invention are therefore directed to such conjugates as well as to compositions comprising the conjugates and related methods as described herein, which are believed to be new and completely unsuggested by the art.

### SUMMARY OF THE INVENTION

Accordingly, a conjugate is provided, the conjugate comprising an hGH moiety covalently attached, either directly or through a spacer moiety, to a nonpeptidic water-soluble polymer. The conjugate is typically although not necessarily provided as part of a composition.

In one aspect of the invention, a conjugate is provided, the conjugate comprising the following structure: 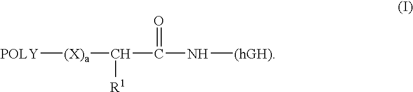
In structure (I), POLY is a water-soluble polymer; (a) is either zero or one; X, when present, is a spacer moiety comprised of one or more atoms; R¹ is an organic radical containing 1 to 3 carbon atoms; and hGH is a residue of a human growth hormone (“hGH”) moiety.

In one or more preferred embodiments, POLY is non-branched, e.g., a linear polymer.

In reference to the conjugates provided herein, POLY is preferably a polyalkylene oxide, and even more preferably, is a poly(ethylene glycol). In one or more particular embodiments, the poly(ethylene glycol) is terminally capped with an end-capping moiety such as methoxy.

In one or more embodiments of the invention, the water-soluble polymer, e.g., poly(ethylene glycol), has a weight average molecular weight in the range of about 130 Daltons to about 110,000 Daltons. Preferably, the water-soluble polymer has a weight average molecular weight in the range of about 6,000 Daltons to about 100,000 Daltons, more preferably from about 10,000 Daltons to about 85,000 Daltons, and most preferably from about 20,000 Daltons to about 65,000 Daltons. Particularly preferred weight average molecular weights of POLY include the following, in Daltons: 5,000; 10,000; 20,000; 25,000; 30,000; 40,000; 45,000; 50,000; and 60,000.

In one or more embodiments of the invention, the hGH moiety is human growth hormone or a biologically active fragment, deletion variant, substitution variant or addition variant thereof.

In one or more preferred embodiments, the hGH moiety is human growth hormone.

In yet another embodiment, the hGH moiety comprises the amino acid sequence of SEQ ID NO:1.

In one or more further embodiments of the invention, the conjugate comprises the following structure: 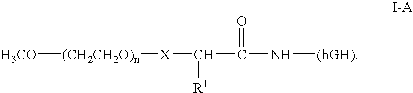
In structure I-A above, (n) is an integer having a value of from 3 to 2500; X is an acyclic spacer moiety comprising from 1 to about 25 atoms; R¹ is an organic radical containing 1 to 3 carbon atoms selected from the group consisting of methyl, ethyl, propyl, and isopropyl; and hGH is a residue of a human growth hormone moiety. Preferably, (n) is an integer having a value of from 150 to 2200.

In one or more particular embodiments of structure I-A, X is an alkylene chain comprising from 1 to 10 carbon atoms.

In yet another particular embodiment, a conjugate of the invention comprises the following structure: 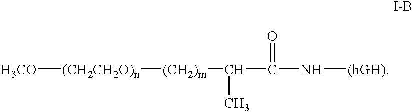
where (m) is an integer ranging from 1 to 8, (n) is an integer having a value from 150 to 2200, and hGH is a residue of a human growth hormone (“hGH”) moiety.

Yet in one or more further embodiments, a conjugate of the invention comprises the following structure: 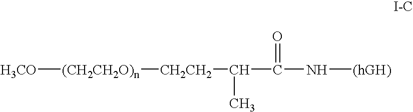
where (n) and hGH are as previously described.

In one or more embodiments, a conjugate of the invention is a monoconjugate or monomer, i.e., is in monoPEGylated form.

In yet one or more alternative embodiments, a conjugate of the invention is a diconjugate or dimer, i.e., is in diPEGylated form.

In yet another aspect, provided is a conjugate comprising the following structure: 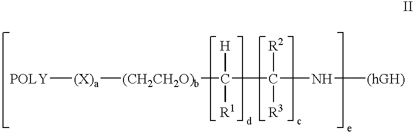
where POLY is a water-soluble polymer; (a) is either zero or one; (b) is either zero or an integer having a value from 1 to 10; (c) is an integer having a value from 1 to 10; (d) is either zero or one; (e) is either one or two; and X, when present, is a spacer moiety comprised of one or more atoms. R¹ is H or an organic radical containing from 1 to 3 carbon atoms. Either R² and R³, when taken together with the carbon atom to which they are attached, in each occurrence considered separately, form a carbonyl (C═O), or R², in each occurrence, is independently H or an organic radical, and R³, in each occurrence, is independently H or an organic radical. hGH is a residue of a human growth hormone moiety.

In one or more embodiments of the invention, one or more of the following provisos may apply to structure II above:

(i) when R² and R³, when taken together with the carbon atom to which they are attached form a carbonyl adjacent to the —NH—, and POLY is linear, then (d) equals one and R¹ is an organic radical containing from 1 to 3 carbon atoms;

(ii) when R² and R³, in each occurrence, are each independently H or an organic radical, then (b) is a positive integer and (a) is one; and

(iii) when POLY is branched, POLY does not contain a lysine residue.

In yet one or more additional embodiments, all of the foregoing provisos (i)-(iii) apply to structure II above.

In yet one or more additional embodiments, provided is a composition comprising one or more conjugates as embodied by the foregoing structures I, I-A, I-B, I-C, and II.

In a particular embodiment, provided is a composition comprising a polymer of structure II where each of provisos (i)-(iii) applies, and further wherein when (e) is one, the composition comprises 85% or greater monoconjugate with respect to all conjugate species contained in the composition, and when (e) is two, the composition comprises greater than 80% diconjugate with respect to all conjugate species contained in the composition, and said composition is bioactive.

In one or more particular embodiments of structure II, (e) is one.

In one or more particular embodiments of structure II, (e) is two.

In reference to structure II, and compositions thereof, the invention encompasses one or more embodiments having one or more of the following features.

In one or more such embodiments, the acyclic spacer moiety, X, comprises from 1 to about 50 atoms, or more preferably, from about 1 to about 25 atoms.

In yet one or more additional embodiments of structure II, R² and R³, when taken together with the carbon atom to which they are attached, form a carbonyl adjacent to the —NH— and POLY is branched or linear. Preferably, POLY is branched. In yet another embodiment of structure II, (b) and (d) both equal zero, and (c) equals 4.

In yet another one or more related embodiments, a composition or conjugate of the invention comprises: 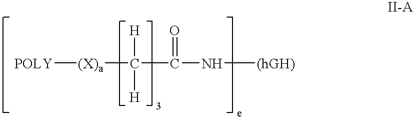
where (a) and (e) are as previously defined for structure II. In one or more embodiments, (a) is zero.

In yet one or more embodiments of structure II or II-A, when POLY is branched, POLY comprises the following structure: 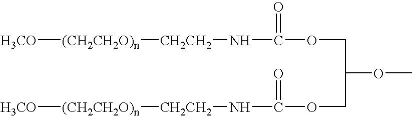
wherein each (n) is independently an integer having a value of from 3 to 2500, more preferably from 150 to 2200.

According to yet another embodiment in reference to structure II and compositions thereof, POLY is branched, (a) is one, (b) ranges from 1 to 10, (c) ranges from 1 to 6, and (d) is zero. In one such preferred embodiment, (b) is 4. In yet one or more additional embodiments, (c) is 4 and R² and R³ are both H in each occurrence. In yet one or more related embodiments, X comprises —CH₂)₃—C(O)—NH—. In yet another related embodiment, POLY comprises the structure: 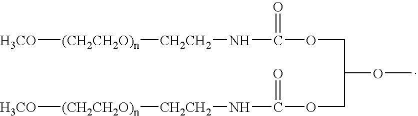
where each (n) is independently an integer having a value of from 150 to 2200.

According to yet one or more additional embodiments in reference to structure II and compositions thereof, POLY is linear, (a) is one, (b) ranges from 1 to 10, (c) ranges from 1-6, and (d) is zero. In a preferred embodiment thereof, (b) is 4. In yet another related embodiment, (c) is 4, and R² and R³ are both H in each occurrence. In yet another related embodiment thereof, X preferably comprises —C(O)—NH—. Even more particularly, in a preferred embodiment, POLY is H₃CO—(CH₂CH₂O)ₙ— and (n) is an integer having a value of from 150 to 2200.

Also provided are compositions comprising any one or more of the foregoing hGH conjugates in combination with a pharmaceutical excipient.

In one or more embodiments, an hGH conjugate-comprising composition of the invention is substantially free of albumin.

In yet another embodiment, an hGH conjugate-comprising composition of the invention is substantially free of proteins that do not possess hGH activity, and/or is substantially free of noncovalently attached water-soluble polymers.

In yet one or more additional embodiments, an hGH conjugate-comprising composition is one where the amount of monoconjugate and diconjugate in the composition represents at least about 85% of the total conjugate species in the composition. In a preferred embodiment, the amount of monoconjugate and diconjugate in the composition represents at least about 90% of the total conjugate species in the composition, and in a most preferred embodiment, the amount of monoconjugate and diconjugate in the composition represents at least about 95% of the total conjugate species in the composition.

In yet another aspect of the invention, provided is a method for preparing a conjugate as described herein. The method comprises the step of contacting one or more polymeric reagents with an hGH moiety under conditions sufficient to result in a conjugate comprising an hGH moiety covalently attached, either directly or through a spacer moiety comprised of one or more atoms, to a water-soluble polymer. The compositions encompass all types of formulations and in particular those that are suited for injection such as powders that can be reconstituted, as well as liquids (e.g., suspensions and solutions).

In yet another aspect, provided is a method for treating a subject for a condition responsive to treatment with human growth hormone, where the method comprises administering to the subject a therapeutically effective amount of an hGH conjugate or composition as described herein. Preferred conjugates and compositions are encompassed by any one or more of the foregoing structures. Conditions that may be treated by administering a conjugate or composition as described herein include growth hormone deficiency, Turner's syndrome, growth failure in pediatric subjects who are born short for gestational age, chronic renal insufficiency, Prader-Willi syndrome, AIDS wasting, and aging. The step of administering the conjugate can be effected by injection (e.g., intramuscular injection, intravenous injection, subcutaneous injection, and so forth), or by other modes (e.g., oral administration).

In one or more particular embodiments of the method, the subject is a pediatric subject, i.e., under 18 years of age.

The invention further encompasses use of a therapeutically effective amount of a conjugate or composition as described herein for treating a mammal suffering from a condition responsive to treatment with human growth hormone.

In one or more particular embodiments, the invention encompasses use of a therapeutically effective amount of a conjugate or composition as described herein for treating growth hormone deficiency.

Also provided is the use of a conjugate or composition as described herein in the preparation of a medicament for administering to a mammal in a therapeutically effective amount for the treatment of a condition responsive to treatment with human growth hormone.

Each of the herein-described features of the invention is meant to apply equally to each and every embodiment as described herein, unless otherwise indicated.

Additional objects, advantages and novel features of the invention will be set forth in the description that follows, and in part, will become apparent to those skilled in the art upon reading the following, or may be learned by practice of the invention.

### BRIEF DESCRIPTION OF THE FIGURES

FIG. 1 is a reproduction of the SDS-PAGE analysis of various conjugates as described in Examples 1 to 4. Lane 1 corresponds to mPEG-2-ButyrALD-40 kD rhGH (Example 4); Lane 2 corresponds to mPEG-2-NHS-40 kD rhGH (Example 1); Lane 3 corresponds to mPEG-ButyrALD-30 kD rhGH (Example 3); Lane 4 corresponds to mPEG-SMB-30 kD rhGH (Example 2); and Lane 5 corresponds to a rhGH stock solution; and Lane 6 corresponds to a Sigma molecular weight marker.

FIGS. 2A-2D correspond to the following chromatograms. FIG. 2A is a reproduction of a size exclusion chromatography (SEC)-HPLC chromatagram of an exemplary hGH conjugate reaction mixture prepared by reacting hGH with mPEG-2-NHS, as described in greater detail in Example 1. FIG. 2B shows the preparative profile of the reaction mixture shown in FIG. 2A following purification by anion-exchange chromatography. FIG. 2C is a reproduction of the SEC-HPLC chromatogram of a purified monoPEGylated hGH conjugate (“1-mer”), designated “mono(mPEG-2-NHS-40 k) hGH”, as described in greater detail in Example 1. FIG. 2D is a reproduction of the SEC-HPLC chromatogram of a purified diPEGylated conjugate (“2-mer”), designated “di(mPEG-2-NHS-40 k) hGH”, as described in greater detail in Example 1.

FIG. 3 is a reproduction of the SEC-HPLC chromatogram of an exemplary conjugate reaction mixture prepared by reacting hGH with mPEG-SMB, as described in greater detail in Example 2.

FIG. 4 is a reproduction of the SEC-HPLC chromatogram of an exemplary conjugate solution prepared by reacting hGH with mPEG-ButyrALD, as described in greater detail in Example 3.

FIG. 5 is a reproduction of the SEC-HPLC chromatogram of an exemplary conjugate solution prepared by reacting hGH with mPEG2-ButyrALD, as described in greater detail in Example 4.

FIG. 6 is a plot demonstrating weight gain in rats following day 8 for six different groups dosed with either rhGH, mono(mPEG-2-NHS-40 k) hGH, di(mPEG-2-NHS-40 k) hGH, or placebo, in different dosing regimes as described in Example 9; and

FIG. 7 is a plot demonstrating weight gain in rats over an eight day time course for six different groups dosed with either rhGH, mono(mPEG-2-NHS-40 k) hGH, di(mPEG-2-NHS-40 k) hGH, or placebo, in different dosing regimens as described in Example 9.

### DETAILED DESCRIPTION OF THE INVENTION

Before describing one or more embodiments of the present invention in detail, it is to be understood that this invention is not limited to the particular polymers, synthetic techniques, hGH moieties, and the like, as such may vary.

Definitions

It must be noted that, as used in this specification and the intended claims, the singular forms “a,” “an,” and “the” include plural referents unless the context clearly dictates otherwise. Thus, for example, reference to “a polymer” includes a single polymer as well as two or more of the same or different polymers, reference to “an optional excipient” refers to a single optional excipient as well as two or more of the same or different optional excipients, and the like.

In describing and claiming one or more embodiments of the present invention, the following terminology will be used in accordance with the definitions described below.

“PEG,” “polyethylene glycol” and “poly(ethylene glycol)” as used herein, are interchangeable and meant to encompass any nonpeptidic water-soluble poly(ethylene oxide). Typically, PEGs for use in accordance with the invention comprise the following structure “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. Throughout the specification and claims, it should be remembered that the term “PEG” includes structures having various terminal or “end capping” groups and so forth. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below.

The terms “end-capped” and “terminally capped” are interchangeably used herein to refer to a terminal or endpoint of a polymer having an end-capping moiety. Typically, although not necessarily, the end-capping moiety comprises a hydroxy or C₁₋₂₀ alkoxy group, more preferably a C₁₋₁₀ alkoxy group, and still more preferably a C₁₋₅ alkoxy group. Thus, examples of end-capping moieties include alkoxy (e.g., methoxy, ethoxy and benzyloxy), as well as aryl, heteroaryl, cyclo, heterocyclo, and the like. It must be remembered that the end-capping moiety may include one or more atoms of the terminal monomer in the polymer [e.g., the end-capping moiety “methoxy” in CH₃O(CH₂CH₂O)ₙ— and CH₃(OCH₂CH₂)ₙ—]. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polymer and/or the moiety (e.g., active agent) to which the polymer is coupled can be determined by using a suitable detector. Such labels include, without limitation, fluorescers, chemiluminescers, moieties used in enzyme labeling, colorimetric (e.g., dyes), metal ions, radioactive moieties, and the like. Suitable detectors include photometers, films, spectrometers, and the like. The end-capping group can also advantageously comprise a phospholipid. When the polymer has an end-capping group comprising a phospholipid, unique properties are imparted to the polymer and the resulting conjugate. Exemplary phospholipids include, without limitation, those selected from the class of phospholipids called phosphatidylcholines. Specific phospholipids include, without limitation, those selected from the group consisting of dilauroylphosphatidylcholine, dioleylphosphatidylcholine, dipalmitoylphosphatidylcholine, disteroylphosphatidylcholine, behenoylphosphatidylcholine, arachidoylphosphatidylcholine, and lecithin.

“Non-naturally occurring” with respect to a polymer as described herein, means a polymer that in its entirety is not found in nature. A non-naturally occurring polymer of the invention may, however, contain one or more monomers or segments of monomers that are naturally occurring, so long as the overall polymer structure is not found in nature.

The term “water soluble” as in a “water-soluble polymer” polymer is any polymer that is soluble in water at room temperature. Typically, a water-soluble polymer will transmit at least about 75%, more preferably at least about 95%, of light transmitted by the same solution after filtering. On a weight basis, a water-soluble polymer will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer is about 95% (by weight) soluble in water or completely soluble in water.

Molecular weight in the context of a water-soluble polymer of the invention, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight. The polymers of the invention are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

The term “active” or “activated” when used in conjunction with a particular functional group, refers to a reactive functional group that reacts readily with an electrophile or a nucleophile on another molecule. This is in contrast to those groups that require strong catalysts or highly impractical reaction conditions in order to react (i.e., a “non-reactive” or “inert” group).

As used herein, the term “functional group” or any synonym thereof is meant to encompass protected forms thereof as well as unprotected forms.

The terms “spacer moiety,” “linkage” or “linker” are used herein to refer to an atom or a collection of atoms optionally used to link interconnecting moieties such as a terminus of a polymer segment and an hGH moiety or an electrophile or nucleophile of an hGH moiety. The spacer moiety may be hydrolytically stable or may include a physiologically hydrolyzable or enzymatically degradable linkage.

“Alkyl” refers to a hydrocarbon chain, typically ranging from about 1 to 15 atoms in length. Such hydrocarbon chains are preferably but not necessarily saturated and may be branched or straight chain, although typically straight chain is preferred. Exemplary alkyl groups include methyl, ethyl, propyl, butyl, pentyl, 1-methylbutyl, 1-ethylpropyl, 3-methylpentyl, and the like. As used herein, “alkyl” includes cycloalkyl as well as cycloalkylene-containing alkyl.

“Lower alkyl” refers to an alkyl group containing from 1 to 6 carbon atoms, and may be straight chain or branched, as exemplified by methyl, ethyl, n-butyl, i-butyl, and t-butyl.

“Cycloalkyl” refers to a saturated or unsaturated cyclic hydrocarbon chain, including bridged, fused, or spiro cyclic compounds, preferably made up of 3 to about 12 carbon atoms, more preferably 3 to about 8 carbon atoms. “Cycloalkylene” refers to a cycloalkyl group that is inserted into an alkyl chain by bonding of the chain at any two carbons in the cyclic ring system.

“Alkoxy” refers to an —O—R group, wherein R is alkyl or substituted alkyl, preferably C₁₋₆ alkyl (e.g., methoxy, ethoxy, propyloxy, and so forth).

The term “substituted” as in, for example, “substituted alkyl,” refers to a moiety (e.g., an alkyl group) substituted with one or more noninterfering substituents, such as, but not limited to: alkyl, C₃₋₈ cycloalkyl, e.g., cyclopropyl, cyclobutyl, and the like; halo, e.g., fluoro, chloro, bromo, and iodo; cyano; alkoxy, lower phenyl; substituted phenyl; and the like. “Substituted aryl” is aryl having one or more noninterfering groups as a substituent. For substitutions on a phenyl ring, the substituents may be in any orientation (i.e., ortho, meta, or para).

“Noninterfering substituents” are those groups that, when present in a molecule, are typically nonreactive with other functional groups contained within the molecule.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl.

“Heteroaryl” is an aryl group containing from one to four heteroatoms, preferably sulfur, oxygen, or nitrogen, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom that is not a carbon. Preferred heteroatoms include sulfur, oxygen, and nitrogen.

“Substituted heteroaryl” is heteroaryl having one or more noninterfering groups as substituents.

“Substituted heterocycle” is a heterocycle having one or more side chains formed from noninterfering substituents.

“Electrophile” and “electrophilic group” refer to an ion or atom or collection of atoms, that may be ionic, having an electrophilic center, i.e., a center that is electron seeking, capable of reacting with a nucleophile.

“Nucleophile” and “nucleophilic group” refer to an ion or atom or collection of atoms that may be ionic having a nucleophilic center, i.e., a center that is seeking an electrophilic center or with an electrophile.

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

A “hydrolytically stable” linkage or bond refers to a chemical bond, typically a covalent bond, that is substantially stable in water, that is to say, does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethanes, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks.

“Pharmaceutically acceptable excipient” or “carrier” refers to an excipient that may optionally be included in the compositions of the invention and that causes no significant adverse toxicological effects to the patient. “Pharmacologically effective amount,” “physiologically effective amount,” and “therapeutically effective amount” are used interchangeably herein to mean the amount of a polymer-(hGH) moiety conjugate that is needed to provide a desired level of the conjugate (or corresponding unconjugated hGH moiety) in the bloodstream or in the target tissue. The precise amount will depend upon numerous factors, e.g., the particular hGH moiety, the components and physical characteristics of the therapeutic composition, intended patient population, individual patient considerations, and the like, and can readily be determined by one skilled in the art, based upon the information provided herein.

“Multi-functional” means a polymer having three or more functional groups contained therein, where the functional groups may be the same or different. Multi-functional polymeric reagents of the invention will typically contain from about 3-100 functional groups, or from 3-50 functional groups, or from 3-25 functional groups, or from 3-15 functional groups, or from 3 to 10 functional groups, or will contain 3, 4, 5, 6, 7, 8, 9 or 10 functional groups within the polymer backbone.

The term “hGH moiety,” as used herein, refers to a moiety having hGH activity. The hGH moiety will also have at least one electrophilic group or nucleophilic group suitable for reaction with a polymeric reagent. In addition, the term “hGH moiety” encompasses both the hGH moiety prior to conjugation as well as the hGH moiety residue following conjugation. As will be explained in further detail below, one of ordinary skill in the art can determine whether any given moiety has hGH activity. A protein comprising an amino acid sequence corresponding to any one of SEQ ID NOS: 1 through 4 is an hGH moiety, as well as any protein or polypeptide substantially homologous thereto, whose biological properties result in the stimulation of growth and/or similar activity of hGH. As used herein, the term “hGH moiety” includes such proteins modified deliberately, as for example, by site directed mutagenesis or accidentally through mutations. These terms also include analogs having from 1 to 6 glycosylation sites, analogs having at least one additional amino acid at the carboxy terminal end of the protein wherein the additional amino acid(s) includes at least one glycosylation site, and analogs having an amino acid sequence which includes at least one glycosylation site. These terms include both natural and recombinantly produced hGH.

The term “substantially homologous” means that a particular subject sequence, for example, a mutant sequence, varies from a reference sequence by one or more substitutions, deletions, or additions, the net effect of which does not result in an adverse functional dissimilarity between the reference and subject sequences. For purposes of the present invention, sequences having greater than 95 percent homology, equivalent biological properties, and equivalent expression characteristics are considered substantially homologous. For purposes of determining homology, truncation of the mature sequence should be disregarded. Sequences having lesser degrees of homology, comparable bioactivity, and equivalent expression characteristics are considered substantial equivalents.

The term “fragment” of an hGH protein means any protein or polypeptide having the amino acid sequence of a portion or fragment of an hGH protein, and which has the biological activity of hGH. Fragments include proteins or polypeptides produced by proteolytic degradation of an hGH protein or produced by chemical synthesis by methods routine in the art. An hGH protein or fragment thereof is biologically active when administration of the protein or fragment to a human results in some degree of growth hormone activity. Determining such biological activity of the hGH protein can carried out by conventional, well known tests utilized for such purposes either in vitro (using, e.g., a receptor binding assay or a cell proliferation assay), or on one or more species of mammals, e.g., using an in vivo rat model. Such assays are described in, e.g., Biochem Biophys Res Commun, 1986, Jan. 14, 134(1), 159-65; Kotts, C. E., et al., Pharmacopeial Forum, U.S. Pharmacopeia, Vol. 25, No. 3, May-June 1999; Groesbeck, M D., Parlow, A F., Endocrinology, 1987, June 120(6): 2582-90; Noble, R. L., et al., Int. J. Pept. Protein Res., 1977, Nov. 10(5), 385-93. An appropriate test that can be utilized to demonstrate such biological activity is described herein.

A “deletion variant” of an hGH moiety is a peptide or protein in which one amino acid residue of the hGH moiety has been deleted and the amino acid residues preceding and following the deleted amino acid residue are connected via an amide bond (except in instances where the deleted amino acid residue was located on a terminus of the peptide or protein). Deletion variants include instances where only a single amino acid residue is deleted, as well as instances where two amino acids are deleted, three amino acids are deleted, four amino acids are deleted, and so forth. Each deletion variant must, however, retain some degree of hGH activity.

A “substitution variant” of an hGH moiety is a peptide or protein in which one amino acid residue of the hGH moiety has been deleted and a different amino acid residue has taken its place. Substitution variants include instances where only a single amino acid residue is substituted, as well as instances where two amino acids are substituted, three amino acids are substituted, four amino acids are substituted, and so forth. Each substitution variant must, however, have some degree of hGH activity.

An “addition variant” of an hGH moiety is a peptide or protein in which one amino acid residue of the hGH moiety has been added into an amino acid sequence and adjacent amino acid residues are attached to the added amino acid residue by way of amide bonds (except in instances where the added amino acid residue is located on a terminus of the peptide or protein, wherein only a single amide bond attaches the added amino acid residue). Addition variants include instances where only a single amino acid residue is added, as well as instances where two amino acids are added, three amino acids are added, four amino acids are added, and so forth. Each addition variant must, however, have some degree of hGH activity.

The term “patient,” refers to a living organism suffering from or prone to a condition that can be prevented or treated by administration of an active agent (e.g., conjugate), and includes both humans and animals.

“Optional” or “optionally” means that the subsequently described circumstance may or may not occur, so that the description includes instances where the circumstance occurs and instances where it does not.

“Substantially” means nearly totally or completely, for instance, satisfying one or more of the following: greater than 50%, 51% or greater, 75% or greater, 80% or greater, 90% or greater, and 95% or greater of the condition.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

Turning to one or more aspects or embodiments of the invention, a conjugate is provided, the conjugate comprising an hGH moiety covalently attached, either directly or through a spacer moiety, to a nonpeptidic water-soluble polymer. The conjugates of the invention possess one or more of the following features.

The hGH Moiety

As previously stated, a conjugate of the invention comprises an hGH moiety covalently attached, either directly or through a spacer moiety, to a nonpeptidic water-soluble polymer. As used herein, the term “hGH moiety” refers to the hGH moiety prior to conjugation as well as to the hGH moiety following attachment to a nonpeptidic water-soluble polymer. It is understood, however, that when the hGH moiety is attached to a nonpeptidic water-soluble polymer, the hGH moiety is slightly altered due to the presence of one or more covalent bonds associated with linkage to the polymer. Often, this slightly altered form of the hGH moiety resulting from attachment to another molecule is referred to a “residue” of the hGH moiety. The hGH moiety in the conjugate is any peptide that provides a growth hormone effect.

The hGH moiety can be derived from either non-recombinant methods or from recombinant methods and the invention is not limited in this regard. In addition, the hGH moiety can be derived from human sources or from animal sources.

The hGH moiety can be derived non-recombinantly. For example, pituitary glands represent a non-recombinant source of hGH moieties. As described in U.S. Pat. No. 2,974,088, homogenized pituitary glands can be treated with an extraction medium (such as acetone), which, in turn, is extracted with an aqueous salt solution. Growth hormone is obtained by precipitation caused by the addition of a suitable miscible organic solvent at alkaline and then acidic pH treatments. Bovine growth hormone, derived from the anterior lobes of bovine pituitaries, can also be used as the hGH moiety. As described in U.S. Pat. No. 3,118,815, chymotrypsin and acid treatments can be used to obtain bovine growth hormone. Other well-known methods for deriving non-recombinant forms of the hGH moiety may also be used.

Alternatively, the hGH moiety can be obtained from recombinant methods. For example, U.S. Pat. No. 4,342,832 describes recombinant-based methods for preparing methionyl hGH (i.e., hGH to which the N-terminus has the amino acid, methionine, attached). In addition, U.S. Pat. No. 5,633,352 describes recombinant methods for preparing hGH. The production of recombinant hGH in E. coli is also described in detail in Jensen, E. Bech, Carlsen, S., Biotechnology and Bioengineering, 36(1), 1990, 1-11. Recombinant hGH is also available from commercial sources, e.g., ProSpec-Tany TechnoGene, Ltd.

The amino acid sequence for human hGH is provided in SEQ ID NO: 1. A methionine residue-containing form of hGH is provided in SEQ ID NO: 2. SEQ ID NO 3 corresponds to SEQ ID NO: 1 wherein a serine residue replaces the phenylalanine residue at position 92, and SEQ ID NO: 4 corresponds to SEQ ID NO: 2 wherein a serine residue replaces the phenylalanine residue at position 93. The hGH moiety can be expressed in bacterial (e.g., Escherichia coli), mammalian (e.g., Chinese hamster ovary cells), and yeast (e.g., Saccharomyces cerevisiae) expression systems.

Although recombinant-based methods for preparing proteins can differ, recombinant methods typically involve constructing the nucleic acid encoding the desired polypeptide or fragment, cloning the nucleic acid into an expression vector, transforming a host cell (e.g., plant, bacteria, yeast, transgenic animal cell, or mammalian cell such as Chinese hamster ovary cell or baby hamster kidney cell), and expressing the nucleic acid to produce the desired polypeptide or fragment. Methods for producing and expressing recombinant polypeptides in vitro and in prokaryotic and eukaryotic host cells are known to those of ordinary skill in the art.

To facilitate identification and purification of the recombinant polypeptide, nucleic acid sequences that encode for an epitope tag or other affinity binding sequence can be inserted or added in-frame with the coding sequence, thereby producing a fusion protein comprised of the desired polypeptide and a polypeptide suited for binding. Fusion proteins can be identified and purified by first running a mixture containing the fusion protein through an affinity column bearing binding moieties (e.g., antibodies) directed against the epitope tag or other binding sequence in the fusion proteins, thereby binding the fusion protein within the column. Thereafter, the fusion protein can be recovered by washing the column with the appropriate solution (e.g., acid) to release the bound fusion protein. The recombinant polypeptide can also be identified and purified by lysing the host cells, separating the polypeptide, e.g., by size exclusion chromatography, and collecting the polypeptide. These and other methods for identifying and purifying recombinant polypeptides are known to those of ordinary skill in the art.

In one or more embodiments of the invention, however, it is preferred that the hGH moiety is not in the form of a fusion protein.

Depending on the system used to express proteins having hGH activity, the hGH moiety can be unglycosylated or glycosylated, and either may be used. That is, the hGH moiety can be unglycosylated or the hGH moiety can be glycosylated. In one or more embodiments of the invention, it is preferred that the hGH moiety is not glycosylated.

The hGH moiety can advantageously be modified to include one or more amino acid residues such as, for example, lysine, cysteine and/or arginine, in order to provide facile attachment of the polymer to an atom within the side chain of the amino acid. In addition, the hGH moiety can be modified to include a non-naturally occurring amino acid residue. Techniques for adding amino acid residues and non-naturally occurring amino acid residues are known to those of ordinary skill in the art. Reference is made to the following: Coligan, J. E., et al., Ed., Current Protocols in Protein Science, Ed., 2003, John Wiley & Sons; Ausubel, F. M., Current Protocols in Molecular Biology, 1988, John Wiley & Sons; Wu, R., et al., Eds. Recombinant DNA Methodology, 1^{st} Edition, Academic Press, 1989. In one or more embodiments of the invention, it is preferred that the hGH moiety is not modified to include one or more amino acid residues.

In addition, the hGH moiety can advantageously be modified to include attachment of one or more functional groups (other than through addition of a functional group-containing amino acid residue). For example, the hGH moiety can be modified to include a thiol group. In addition, the hGH moiety can be modified to include an N-terminal alpha carbon. In addition, the hGH moiety can be modified to include one or more carbohydrate moieties. In some embodiments of the invention, it is preferred that the hGH moiety is not modified to include a thiol group and/or an N-terminal alpha carbon.

A preferred hGH moiety has an amino acid sequence selected from the group consisting of SEQ ID NO: 1, SEQ ID NO: 2, SEQ ID NO: 3, and SEQ ID NO: 4. Unless specifically noted, all assignments of a numeric location of an amino acid residue as provided herein are based on SEQ ID NO: 1. Examplary sequences that are useful to serve as hGH moieties include those sequences of the proteins found in commercially available growth hormone formulations such as HUMATROPE® hGH (Eli Lilly and Company, Indianapolis, Ind.), GENOTROPIN® hGH (Pharmacia AB, Stockholm Sweden), and NUTROPIN® hGH (Genentech, Inc., South San Francisco, Calif.).

Although the hGH moiety having an amino acid sequence selected from the group consisting of SEQ ID NO: 1, SEQ ID NO: 2, SEQ ID NO: 3, SEQ ID NO: 4, methionyl hGH, and other growth hormones are useful herein, truncated versions, hybrid variants, and peptide mimetics of any of the foregoing can also be used. Biologically active fragments, deletion variants, substitution variants or addition variants of any of the foregoing that maintain at least some degree of hGH activity can also serve as an hGH moiety.

For any given peptide or protein moiety, it is possible to determine whether that moiety has hGH activity. Determining such biological activity of the hGH protein can carried out by conventional, well-known tests utilized for such purposes. For example, either an in vitro assay (using, e.g., a receptor binding assay or a cell proliferation assay), or an in vivo model (e.g., an in vivo rat model), can be employed. Such assays are described in, e.g., Biochem Biophys Res Commun, 1986, Jan. 14, 134(1), 159-65; Kotts, C. E., et al., Pharmacopeial Forum, US. Pharmacopeia, Vol. 25, No. 3, May-June 1999; Groesbeck, M D., Parlow, A F., Endocrinology, 1987, June 120(6): 2582-90; Noble, R. L., et al., Int. J. Pept. Protein Res., 1977, Nov. 10(5), 385-93. For example, as described in International Publication WO 93/00109, hypophysectomized rats can be anaesthetized and periodically injected with a candidate hGH moiety. The candidate hGH moiety can serve as an hGH moiety in accordance with the present invention if rats injected with the proposed hGH moiety exhibit a statistically significant increase in weight when compared to control rats not injected with the proposed hGH moiety.

The Water-Soluble Polymer

A conjugate of the present invention comprises an hGH moiety covalently attached to a water-soluble polymer. With respect to the water-soluble polymer, the water-soluble polymer is nonpeptidic, non-toxic, non-naturally occurring and biocompatible. With respect to biocompatibility, a substance is considered biocompatible if the beneficial effects associated with use of the substance alone or with another substance (e.g., an active agent such as an hGH moiety) in connection with living tissues (e.g., administration to a patient) outweighs any deleterious effects as evaluated by a clinician, e.g., a physician. With respect to non-immunogenicity, a substance is considered nonimmunogenic if the intended use of the substance in vivo does not produce an undesired immune response (e.g., the formation of antibodies) or, if an immune response is produced, that such a response is not deemed clinically significant or important as evaluated by a clinician. It is particularly preferred that the nonpeptidic water-soluble polymer is biocompatible and non-immunogenic.

Further, the polymer is typically characterized as having from 2 to about 300 termini. Examples of such polymers include, but are not limited to, poly(alkylene glycols) such as polyethylene glycol (PEG), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and combinations of any of the foregoing.

The polymer is not limited to a particular structure and can be linear (e.g., alkoxy PEG or bifunctional PEG), branched or multi-armed (e.g., forked PEG or PEG attached to a polyol core), dendritic, or with degradable linkages. Moreover, the internal structure of the polymer can be organized in any number of different patterns and can be selected from the group consisting of homopolymer, alternating copolymer, random copolymer, block copolymer, alternating tripolymer, random tripolymer, and block tripolymer.

Typically, activated PEG and other activated water-soluble polymers (i.e., polymeric reagents) are activated with a suitable activating group appropriate for coupling to a desired site on the hGH moiety. Thus, a polymeric reagent will possess a reactive group for reaction with the hGH moiety. Representative polymeric reagents and methods for conjugating these polymers to an active moiety are known in the art and further described in, e.g., Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992); Zalipsky (1995) Advanced Drug Reviews 16:157-182; Roberts, M. J., et al., Advanced Drug Delivery Reviews, 54 (2002), 459-476; and Zalipsky, S., Bioconjugate Chemistry, 1995, 6, 150-165.

Typically, the weight-average molecular weight of the water-soluble polymer in the conjugate is from about 100 Daltons to about 150,000 Daltons. Exemplary ranges, however, include weight-average molecular weights in the range of greater than 5,000 Daltons to about 100,000 Daltons, in the range of from about 6,000 Daltons to about 100,000 Daltons, in the range of from about 6,000 to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 20,000 Daltons to about 65,000 Daltons, in the range of from about 25,000 Daltons to about 120,000 Daltons; in the range of from about 25,000 Daltons to about 85,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. For any given water-soluble polymer, PEGs having a molecular weight in one or more of these ranges are preferred.

Exemplary weight-average molecular weights for the water-soluble polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the water-soluble polymer (e.g., a branched 40,000 Dalton water-soluble polymer comprised of two 20,000 Dalton polymers) having a total molecular weight of any of the foregoing can also be used. In one or more embodiments, the conjugate will not have any PEG moieties attached, either directly or indirectly, with a PEG having a weight average molecular weight of less than about 6,000 Daltons.

When used as the polymer, PEGs will typically comprise a number of (OCH₂CH₂) monomers [or (CH₂CH₂O) monomers, depending on how the PEG is defined]. As used throughout the description, the number of repeating units is identified by the subscript “n” in “(OCH₂CH₂)ₙ.” Thus, the value of (n) typically falls within one or more of the following ranges: from 2 to about 3400, from 3 to about 2500, from about 100 to about 2300, from about 100 to about 2270, from about 136 to about 2050, from about 150 to 2200, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

One particularly preferred polymer for use in the invention is an end-capped polymer, that is, a polymer having at least one terminus capped with a relatively inert group, such as a lower C₁₋₆ alkoxy group, although a hydroxyl group can also be used. When the polymer is PEG, for example, it is preferred to use a methoxy-PEG (commonly referred to as mPEG), which is a linear form of PEG wherein one terminus of the polymer is a methoxy (—OCH₃) group, while the other terminus is a hydroxyl or other functional group that can be optionally chemically modified, or is chemically activated, e.g., for conjugation to a protein such as hGH.

In one form useful in one or more embodiments of the present invention, free or unbound PEG is a linear polymer terminated at each end with hydroxyl groups:
HO—CH₂CH₂O—(CH₂CH₂O)ₙ—CH₂CH₂—OH,
wherein (n) typically ranges from zero to about 4,000. The above polymer, alpha-, omega-dihydroxylpoly(ethylene glycol), can be represented in brief form as HO-PEG-OH (also commonly referred to as PEG-diol) where it is understood that the —PEG-symbol can represent the following structural unit:
—CH₂CH₂O—(CH₂CH₂O)ₙ—CH₂CH₂—,
wherein (n) is as defined as above.

Another type of PEG useful in one or more embodiments of the present invention is methoxy-PEG-OH, or mPEG in brief, in which one terminus is the relatively inert methoxy group, while the other terminus is a hydroxyl group. The structure of mPEG is given below.
CH₃O—CH₂CH₂O—(CH₂CH₂O)ₙ—CH₂CH₂—OH
wherein (n) is as described above. Both PEG diol and mPEG can be used for conjugation to a protein such as hGH, or, may be further modified to form a polymeric reagent suitable for covalent attachment to an hGH moiety.

Multi-armed or branched PEG molecules, such as those described in U.S. Pat. No. 5,932,462, can also be used as the PEG polymer. For example, PEG can have the structure: 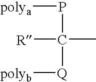
wherein: polyₐ and poly_{b} are PEG backbones (either the same or different), such as methoxy poly(ethylene glycol); R″ is a nonreactive moiety, such as H, methyl or a PEG backbone; and P and Q are nonreactive linkages. In certain embodiments, the branched PEG polymer is methoxy poly(ethylene glycol) disubstituted lysine, or a derivative thereof. In yet other certain embodiments, a branched polymer comprising an hGH conjugate of the invention does not contain a lysine residue. Additional branched polymeric reagents for use in the invention are provided in the tables that follow. Depending on the specific hGH moiety used, the reactive ester functional group of the disubstituted lysine may be further modified to form a different functional group suitable for reaction with a target group within the hGH moiety.

In addition, the PEG can comprise a forked PEG structure. An example of a forked PEG is represented by the following generalized structure: 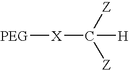
wherein X is a spacer moiety comprised of one or more atoms and each Z is an activated terminal group linked to the —CH by a chain of atoms of defined length. U.S. Pat. No. 6,362,254 describes various forked PEG structures capable of use in one or more embodiments of the present invention. The chain of atoms linking the Z functional groups to the branching carbon atom serve as a tethering group and may comprise, for example, alkyl chains, ether chains, ester chains, amide chains and combinations thereof.

The water-soluble polymer may also comprise a somewhat sterically-hindered architecture, such as the sterically hindered alkanoic acids and alkanoic acid derivatives described in U.S. Pat. No. 6,495,659. Examples of such polymeric reagents include PEG succinimidyl methyl propionate (SMP) and PEG succinimidyl butanoate (SMB).

Also useful are polymer ketone reagents such as those described in U.S. Patent Publication No. 2005/0031576.

The PEG polymer may comprise a pendant PEG molecule having reactive groups, such as carboxyl, covalently attached along the length of the PEG rather than at the end of the PEG chain. The pendant reactive groups can be attached to the PEG directly or through a spacer moiety, such as an alkylene group.

In addition to the above-described forms of PEG, the polymer can also be prepared with one or more weak or degradable linkages in the polymer, including any of the above-described polymers. For example, PEG can be prepared with ester linkages in the polymer that are subject to hydrolysis. As shown below, this hydrolysis results in cleavage of the polymer into fragments of lower molecular weight:
-PEG-CO₂-PEG-+H₂O→-PEG-CO₂H+ HO-PEG-
Other hydrolytically degradable linkages, useful as a degradable linkage within a polymer backbone, include: carbonate linkages; imine linkages resulting, for example, from reaction of an amine and an aldehyde (see, e.g., Ouchi et al. (1997) Polymer Preprints 38(1):582-3); phosphate ester linkages formed, for example, by reacting an alcohol with a phosphate group; hydrazone linkages which are typically formed by reaction of a hydrazide and an aldehyde; acetal linkages that are typically formed by reaction between an aldehyde and an alcohol; orthoester linkages that are, for example, formed by reaction between a formate and an alcohol; amide linkages formed by an amine group, e.g., at an end of a polymer such as PEG, and a carboxyl group of another PEG chain; urethane linkages formed from reaction of, e.g., a PEG with a terminal isocyanate group and a PEG alcohol; peptide linkages formed by an amine group, e.g., at an end of a polymer such as PEG, and a carboxyl group of a peptide; and oligonucleotide linkages formed by, for example, a phosphoramidite group, e.g., at the end of a polymer, and a 5′ hydroxyl group of an oligonucleotide.

The introduction of one or more degradable linkages into the polymer chain may provide for additional control over the final desired pharmacological properties of the conjugate upon administration. For example, a large and relatively inert conjugate (i.e., having one or more high molecular weight PEG chains attached thereto, for example, one or more PEG chains having a molecular weight greater than about 10,000, wherein the conjugate possesses essentially no bioactivity) may be administered, which is hydrolyzed to generate a bioactive conjugate possessing a portion of the original PEG chain. In this way, the properties of the conjugate can be more effectively tailored to balance the bioactivity of the conjugate over time.

The water-soluble polymer associated with the conjugate can also be “cleavable.” That is, the water-soluble polymer cleaves (either through hydrolysis, enzymatic processes, or otherwise), thereby resulting in the unconjugated hGH moiety. In some instances, cleavable polymers detach from the hGH moiety in vivo without leaving any fragment of the water-soluble polymer. In other instances, cleavable polymers detach from the hGH moiety in vivo leaving a relatively small fragment (e.g., a succinate tag) from the water-soluble polymer. An exemplary cleavable polymer includes one that attaches to the hGH moiety via a carbonate linkage, such as those polymeric reagents described in U.S. Pat. No. 6,541,015.

Those of ordinary skill in the art will recognize that the foregoing discussion concerning nonpeptidic and water-soluble polymers is by no means exhaustive and is merely illustrative, and that all polymeric materials having the qualities described above are contemplated. As used herein, the term “polymeric reagent” generally refers to an entire molecule, which can comprise a water-soluble polymer segment and a functional group.

hGH Conjugates

As described above, a conjugate of the invention comprises one or more water-soluble polymer covalently attached (directly or indirectly) to an hGH moiety. Typically, for any given conjugate, there will be one to three water-soluble polymers covalently attached to one or more moieties having hGH activity. In some instances, however, the conjugate may have 1, 2, 3, 4, 5, 6, 7, 8 or more water-soluble polymers individually attached to an hGH moiety. Preferred are hGH conjugates having one or two water-soluble polymers covalently attached thereto.

The particular linkage within the moiety having hGH activity and the polymer depends on a number of factors. Such factors include, for example, the particular linkage chemistry employed, the particular hGH moiety, the available functional groups within the hGH moiety (either for attachment to a polymer or conversion to a suitable attachment site), the presence of additional reactive functional groups within the hGH moiety, and the like.

The conjugates of the invention can be, although are not necessarily, prodrugs, meaning that the linkage between the polymer and the hGH moiety is hydrolytically degradable to allow release of the parent moiety. Exemplary degradable linkages include carboxylate ester, phosphate ester, thiolester, anhydrides, acetals, ketals, acyloxyalkyl ether, imines, orthoesters, peptides and oligonucleotides. Such linkages can be readily prepared by appropriate modification of either the hGH moiety (e.g., the carboxyl group C terminus of the protein or a side chain hydroxyl group of an amino acid such as serine or threonine contained within the protein) and/or the polymeric reagent using coupling methods commonly employed in the art. Most preferred, however, are hydrolyzable linkages that are readily formed by reaction of a polymeric reagent with a non-modified functional group contained within the moiety having hGH activity.

Alternatively, a hydrolytically stable linkage, such as an amide, urethane (also known as carbamate), amine, thioether (also known as sulfide), or urea (also known as carbamide) linkage can also be employed as the linkage for coupling the hGH moiety. Again, preferred hydrolytically stable linkages between the hGH moiety and the polymeric reagent are an amide or a secondary amine linkage. In one approach for preparing an amide-linked conjugate, a water-soluble polymer bearing an activated ester, e.g., mPEG-succinimidyl-α-methylbutanoate or a branched mPEG-N-hydroxysuccinimide, mPEG-2-NHS, is reacted with an amine group on the hGH moiety to thereby result in an amide linkage between the hGH moiety and the water-soluble polymer. Examples of such coupling are provided in Example 1 and Example 2.

In yet an alternative approach, a polymeric reagent containing a reactive aldehyde group, e.g., mPEG butyraldehyde, or a branched mPEG butyraldehyde, as described in Examples 3 and 4, respectively, is reacted with an amine group on an hGH moiety under conditions effective to promote reaction to form an imine-linked intermediate. This imine-linked intermediate is then reduced with an appropriate reducing agent, e.g., sodium cyanoborohydride, sodium borohydride, lithium aluminum hydride, etc., to form a secondary amine-linkage between the hGH moiety and the water-soluble polymer. The selectivity of the reaction is typically adjusted by modification of pH with a non-amine containing buffer. Lower pHs, e.g., typically from about 4 to about 6.5 tend to favor N-terminal conjugation while higher pHs, e.g., typically above about 7.0, favor non-N-terminal conjugation.

The conjugates (as opposed to an unconjugated hGH moiety) may or may not possess a measurable degree of hGH activity. That is to say, a polymer-hGH moiety conjugate in accordance with the invention will typically possess anywhere from about 0.1% to about 100% of the bioactivity of the unmodified parent hGH moiety. In some instances, the polymer-hGH moiety conjugates may possess greater than 100% bioactivity of the unmodified parent hGH moiety. Conjugates possessing little or no hGH activity may contain a hydrolyzable linkage connecting the polymer to the moiety, so that regardless of the lack of activity in the conjugate, the active parent molecule (or a derivative thereof) is released upon aqueous-induced cleavage of the hydrolyzable linkage. However, as can be seen by the results of the preliminary in vitro and in vivo bioassays described in Example 9, although the illustrative conjugates described therein exhibited a significant reduction in activity when compared to unmodified hGH, such reduction in bioactivity can be more than balanced by an increased circulating half-life and/or plasma availability of a suitably-chosen hGH conjugate. This surprising feature lends to another aspect of the present invention, i.e., an hGH conjugate that exhibits a bioactivity of no more than about 25% that of unmodified hGH, yet can be administered in a therapeutically effective amount to a subject in need thereof due to its greatly increased circulating half-life and or plasma availability in comparison to unmodified hGH. Such illustrative conjugates, whose particular structures are provided in greater detail below, can possess no more than about 20% of the bioactivity of unmodified hGH, or even no more than about 15%, or even no more than about 10%, or even no more than about 5%, or even no more than about 3% of the bioactivity of unmodified hGH, yet still be therapeutically effective for the reasons provided above.

Thus, for conjugates possessing a hydrolytically stable linkage that couples the moiety having hGH activity to the polymer, the conjugate will typically possess at least a measurable degree of bioactivity. For instance, such conjugates are typically characterized as having a bioactivity of at least about 0.2%, 0.5%, 1%, 2%, 5%, 10%, 15%, 25%, 30%, 40%, 50%, 60%, 80%, 85%, 90%, 95% 97%, 100%, or more relative to that of the unconjugated hGH moiety, when measured in a suitable model, such as those well known in the art. Preferably, conjugates having a hydrolytically stable linkage (e.g., an amide linkage or a secondary amine linkage) will possess at least some degree of the bioactivity of the unmodified parent moiety having hGH activity.

Exemplary conjugates in accordance with the invention will now be described wherein the hGH moiety is a protein. Typically, such a protein is expected to share (at least in part) a similar amino acid sequence to native human hGH. Thus, while reference will be made to specific locations or atoms within the native human hGH protein, such reference is for convenience only and one having ordinary skill in the art will be able to readily determine the corresponding location or atom in other moieties having hGH activity. In particular, the description provided herein for native human hGH is often applicable to fragments, deletion variants, substitution variants or addition variants of any of the foregoing.

As stated above, amino groups on an hGH moiety provide a point of attachment to the water-soluble polymer. In one embodiment, the conjugate has one water-soluble polymer attached at the N-terminal of the hGH moiety, in some instances, however, the composition will contain less than 50% of N-terminus monoPEGylated conjugates. Human GH comprises nine amine-containing lysine residues and one amino terminus (see SEQ ID NO: 1). Thus, exemplary attachment points of this hGH include attachment at the amine side chain associated with a lysine at any one or more of positions 38, 41, 70, 115, 140, 145, 158, 168 and 172.

There are a number of examples of suitable polymeric reagents useful for forming covalent linkages with available amines of an hGH moiety. Specific examples, along with the corresponding conjugate, are provided in Table 1, below. In the table, the variable (n) represents the number of repeating monomeric units and “(hGH)” represents the residue of the hGH moiety following conjugation to the polymeric reagent. While each polymeric portion [e.g., (OCH₂CH₂)ₙ or (CH₂CH₂O)ₙ] presented in Table 1 terminates in a “CH₃” group, other groups (such as H and benzyl) can be substituted therefor.

As can be seen from the table below, functional groups capable of reacting with either the terminal α-amino group or ε-amino groups of lysines within the hGH moiety include, e.g., N-hydroxysuccinimidyl esters, p-nitrophenylcarbonates, succinimidylcarbonates, aldehydes, acetals, N-keto-piperidones, maleimides, carbonyl imidazoles, azalactones, cyclic imide thiones, isocyanates, isothiocyanates, tresyl chloride, and halogen formates, among others.

TABLE 1
Amine-Specific Polymeric Reagents and the hGH Moiety Conjugate Formed Therefrom
Polymeric Reagent

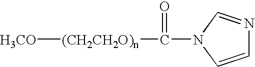

mPEG-Oxycarbonylimidazole Reagent

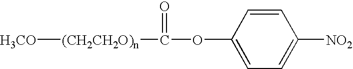

mPEG Nitrophenyl Reagent

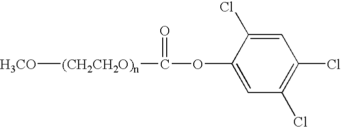

mPEG-Trichlorophenylcarbonate Reagent

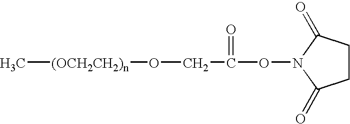

mPEG-Succinimidyl Reagent

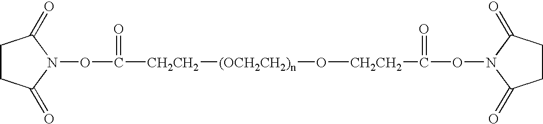

Homobifunctional PEG-Succinimidyl Reagent

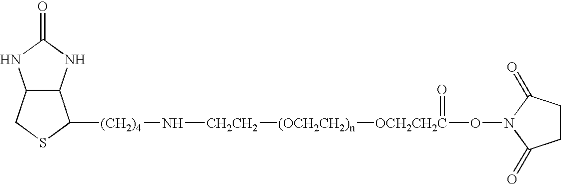

Heterobifunctional PEG-Succinimidyl Reagent

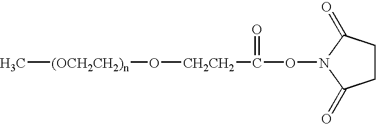

mPEG-Succinimidyl Reagent

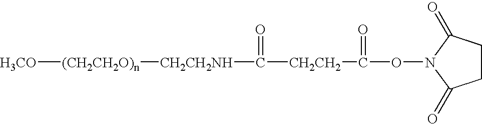

mPEG-Succinimdyl Reagent

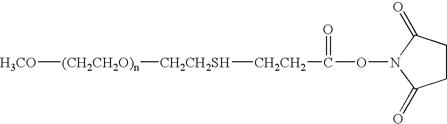

mPEG Succinimidyl Reagent

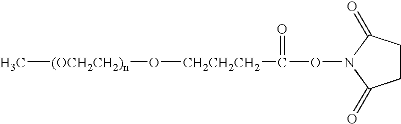

mPEG-Succinimidyl Reagent

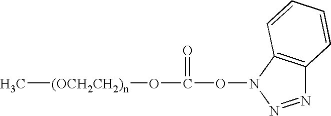

mPEG-Benzotriazole Carbonate Reagent

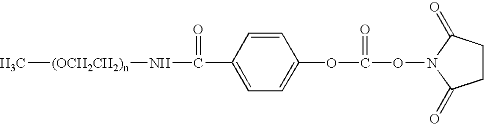

mPEG-Succinimidyl Reagent

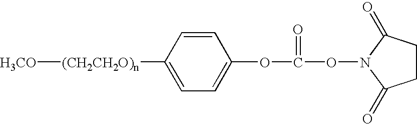

mPEG-Succinimidyl Reagent

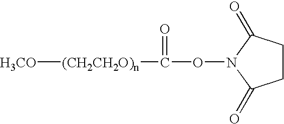

mPEG Succinimidyl Reagent

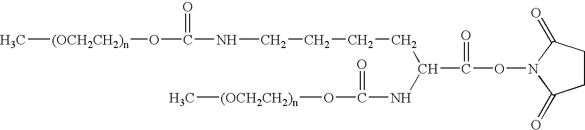

Branched mPEG2-N-Hydroxysuccinimide
Reagent

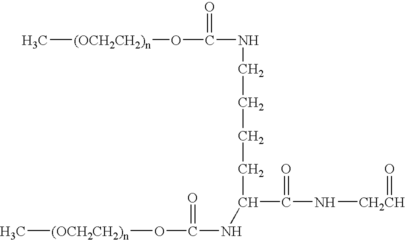

Branched mPEG2-Aldehyde Reagent

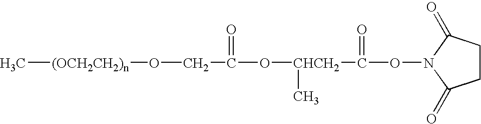

mPEG-Succinimidyl Reagent

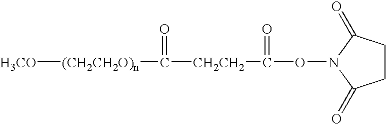

mPEG-Succinimidyl Reagent

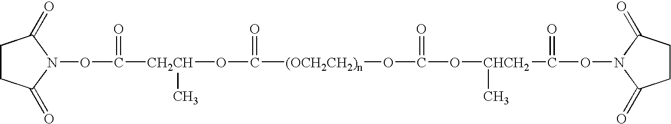

Homobifunctional PEG-Succinimidyl Reagent

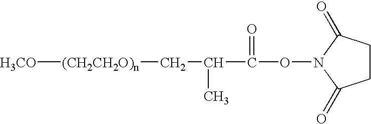

mPEG-Succinimidyl Reagent

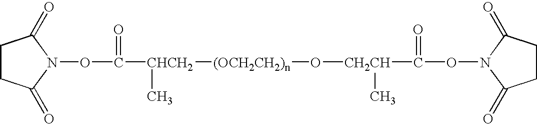

Homobifunctional PEG-Succinimidyl
Propionate Reagent

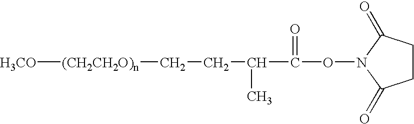

mPEG-Succinimidyl Reagent

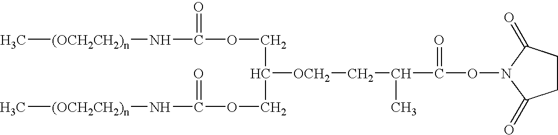

Branched mPEG2-N-Hydroxysuccinimide
Reagent

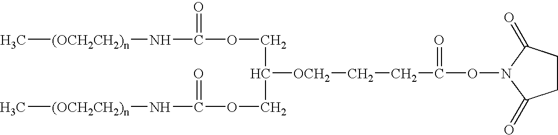

Branched mPEG2-N-Hydroxysuccinimide
Reagent

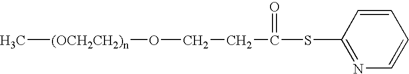

mPEG-Thioester Reagent

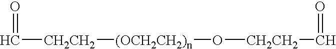

Homobifunctional PEG Propionaldehyde
Reagent

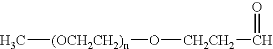

mPEG Propionaldehyde Reagent

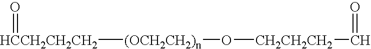

Homobifunctional PEG Butyraldehyde Reagent

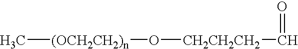

mPEG Butryaldehyde Reagent

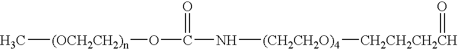

mPEG Butryaldehyde Reagent

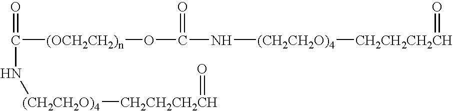

Homobifunctional PEG Butryaldehyde Reagent

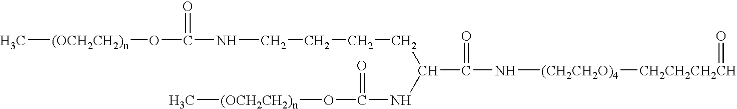

Branched mPEG2 Butyraldehyde Reagent

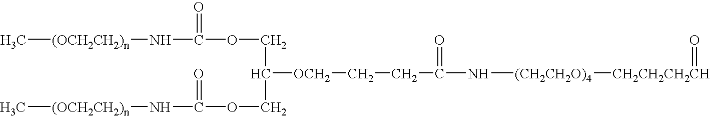

Branched mPEG2 Butyraldehyde Reagent

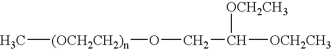

mPEG Acetal Reagent

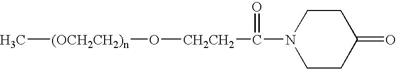

mPEG Piperidone Reagent

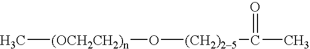

mPEG Methylketone Derivative

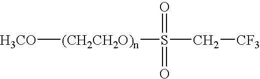

mPEG tresylate

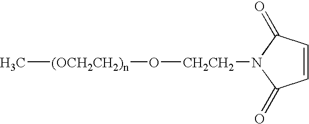

mPEG Maleimide Derivative
(under certain reaction conditions such as pH > 8)

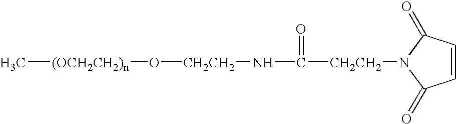

mPEG Maleimide Derivative
(under certain reaction conditions such as pH > 8)

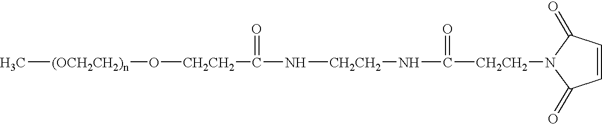

mPEG Maleimide Derivative
(under certain reaction conditions such as pH > 8)

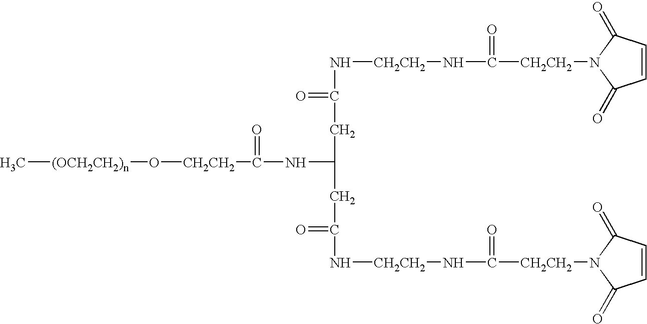

mPEG Forked Maleimide Derivative
(under certain reaction conditions such as pH > 8)

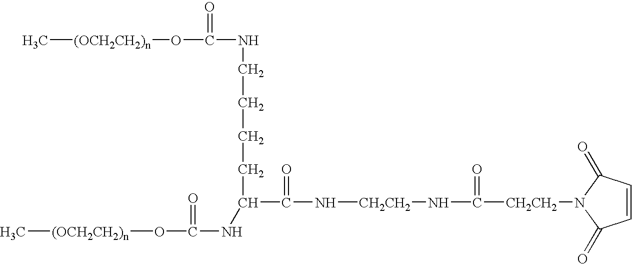

branched mPEG2 Maleimide Derivative
(under certain reaction conditions such as pH > 8)
Amine-Specific Polymeric Reagents and the hGH Moiety Conjugate Formed Therefrom
Corresponding Conjugate

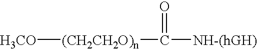

Carbamate Linkage

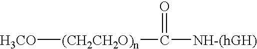

Carbamate Linkage

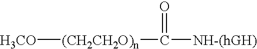

Carbamate Linkage

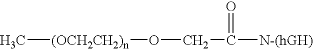

Amide Linkage

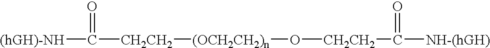

Amide Linkages

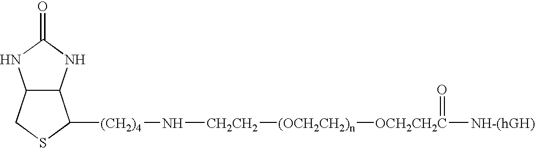

Amide Linkage

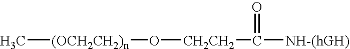

Amide Linkage

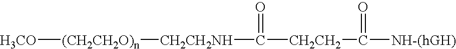

Amide Linkage

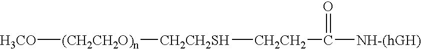

Amide Linkage

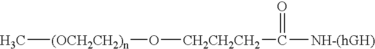

Amide Linkage

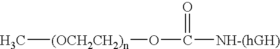

Carbamate Linkage

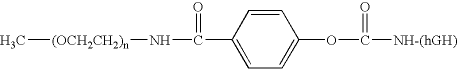

Carbamate Linkage

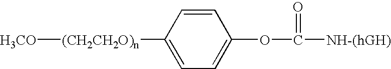

Amide Linkage

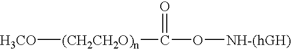

Amide Linkage

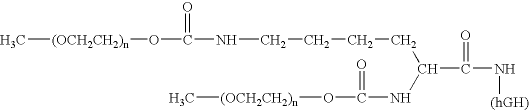

Amide Linkage

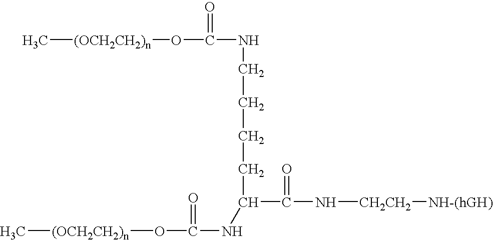

Secondary Amine Linkage

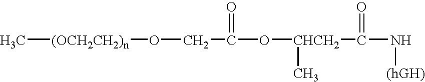

Amide Linkage

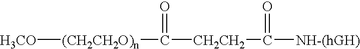

Amide Linkage

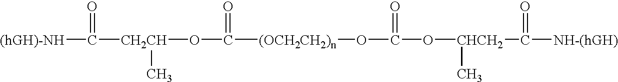

Amide Linkages

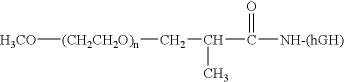

Amide Linkage

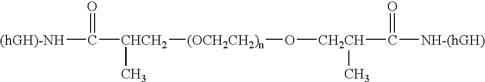

Amide Linkages

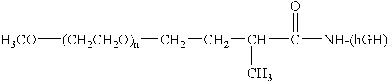

Amide Linkage

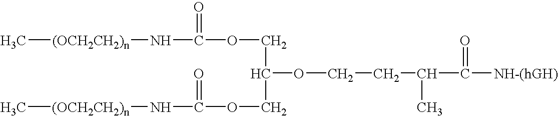

Amide Linkage

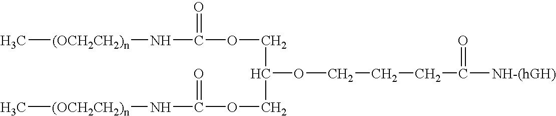

Amide Linkage

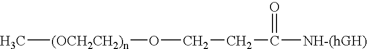

Amide Linkage (typically to hGH moiety
having a N-terminal cysteine or histidine)

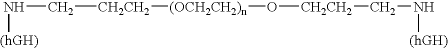

Secondary Amine Linkages

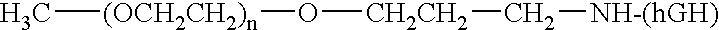

Secondary Amine Linkage

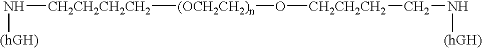

Secondary Amine Linkages

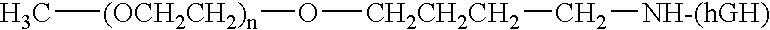

Secondary Amine Linkage

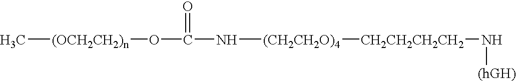

Secondary Amine Linkage

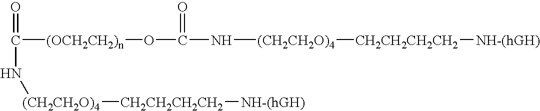

Secondary Amine Linkages

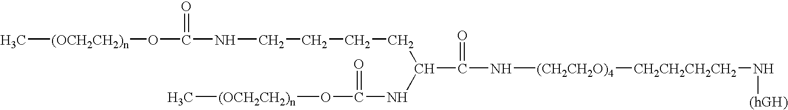

Secondary Amine Linkage

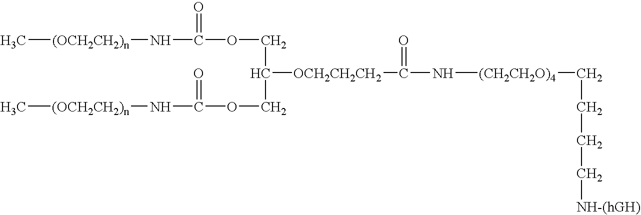

Secondary Amine Linkage

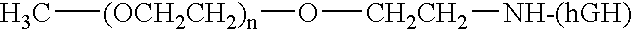

Secondary Amine Linkage

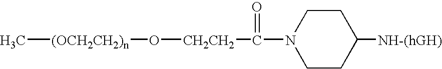

Secondary Amine Linkage
(to a secondary carbon)

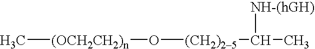

secondary amine linkage
(to a secondary carbon)

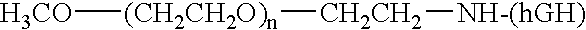

Secondary Amine Linkage

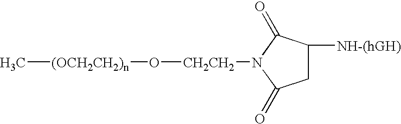

Secondary Amine Linkage

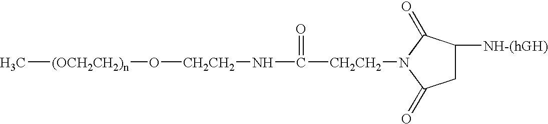

Secondary Amine Linkage

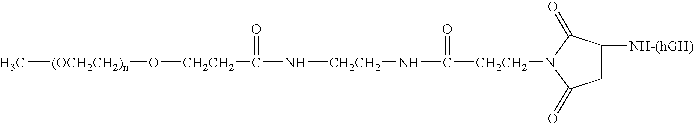

Secondary Amine Linkage

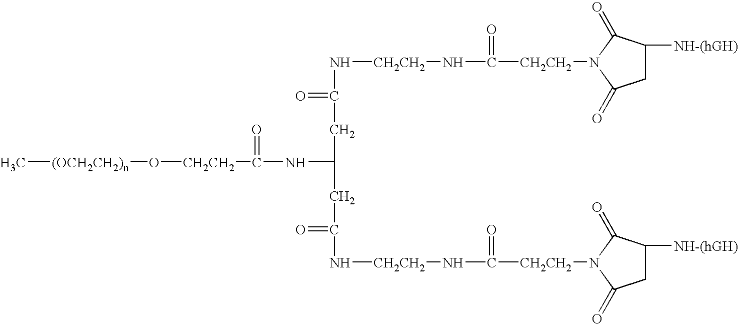

Secondary Amine Linkages

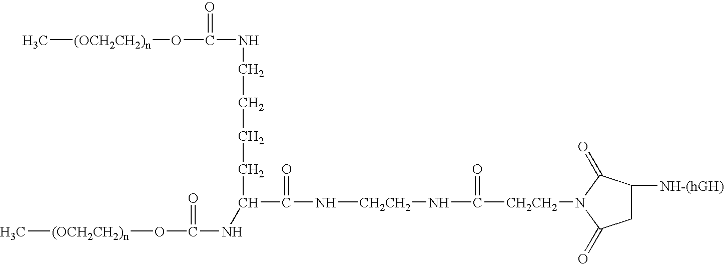

Secondary Amine Linkage

Conjugation of a polymeric reagent to an amino group of an hGH moiety can be accomplished in accordance with the procedures provided herein. In one approach, an hGH moiety is conjugated to a polymeric reagent functionalized with a succinimidyl derivative (or other activated ester group, wherein approaches similar to those described for these alternative activated ester group-containing polymeric reagents can be used). In this approach, the polymer bearing a succinimidyl group is attached to the hGH moiety in an aqueous medium at a pH of about 7 to 9.0 at room temperature, although somewhat modified reaction conditions (e.g., a lower pH such as a pH from about 6 to 7, or a different reaction temperature, e.g., greater or less than room temperature) can also be employed. However, such modified conditions may result in the attachment of the polymer to a different location on the hGH moiety. For instance, at slightly acidic pHs, conjugation at a histidine site is favored, while at higher pHs, covalent attachment at the hydroxyl groups of tyrosine is somewhat favored. Alternatively, an amide linkage can be formed by reacting an amine-terminated nonpeptidic, water-soluble polymer with the carboxy-terminus of an hGH moiety, optionally in activated form.

An exemplary hGH conjugate having an amide linkage covalently linking the hGH moiety with the water soluble polymer in accordance with the invention comprises the following structure: 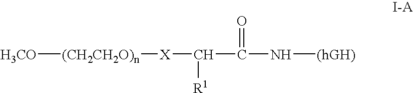
where (n) is an integer having a value of from 3 to 4000, preferably from about 150 to 2200, X is as previously defined; R¹ is an organic radical containing 1 to 3 carbon atom, e.g., such as methyl, ethyl, propyl, and isopropyl; and hGH is a residue of a human growth hormone moiety. Preferably, X is an acyclic spacer moiety containing from about 1 to about 25 atoms. For example, X may be an alkylene chain comprising from 1 to 10 carbon atoms, e.g., —(CH₂)ₘ, where m is an integer ranging from 1 to 10, or from 1 to 8. Thus, the alkylene chain may contain a number of methylenes selected from 1, 2, 3, 4, 5, 6, 7, 8, 9, or 10. Conjugates falling within this structure may possess a weight average molecular weight ranging from about 6,000 to about 100,000 Daltons, or preferably from about 10,000 to about 85,000 Daltons, or even more preferably from about 20,000 to about 65,000 Daltons. Exemplary conjugates may possess a weight average molecular weight of about 20,000 Daltons, or about 30,000 Daltons, or about 40,000 Daltons or greater.

One such conjugate comprises either of the following structures: 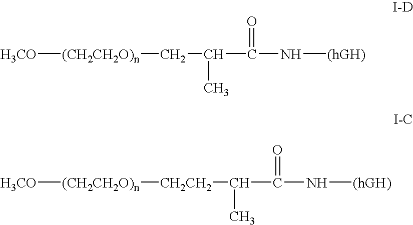
where (n) is an integer having a value of from 3 to 4000, more preferably from 150-2200. The above conjugates are preferably monoPEGylated or diPEGylated conjugates (i.e, having one PEG moiety covalently attached to hGH, or having two PEG moieties covalently attached to hGH).

Additional exemplary conjugates having an amide linkage covalently linking the hGH moiety to the water-soluble polymer may possess one or more of the following structures, wherein POLY is branched. For example, one such illustrative conjugate of the invention comprises the following structure: 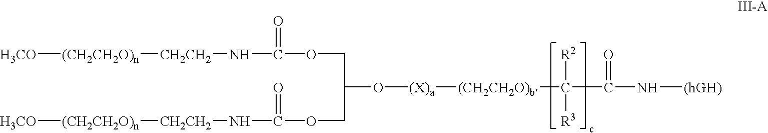
In the preceding structure, each (n) is independently an integer having a value of from 3 to 4000, preferably from 150 to 2200; (a) is either zero or one (meaning that (X) is optionally present); X, when present, is a spacer moiety comprised of one or more atoms; (b′) is zero or an integer having a value of one through ten; (c) is an integer having a value of one through ten; R², in each occurrence, is independently H or an organic radical; R³, in each occurrence, is independently H or an organic radical; and hGH is a residue of human growth hormone moiety.

In yet a more particular embodiment, an exemplary conjugate of the invention comprises the following structure: 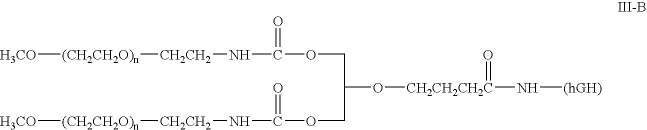
where the values of (n) and hGH are as previously described.

Preferred amine groups in hGH that can serve as a site for attaching a polymer include those amine groups found within a lysine residue. In addition, the N-terminus of any hGH moiety that is a protein can also serve as a polymeric attachment site.

Typical of another approach useful for conjugating the hGH moiety to a polymeric reagent is reductive amination to conjugate a primary amine of an hGH moiety to a polymeric reagent functionalized with a ketone, aldehyde or a hydrated form thereof (e.g., ketone hydrate, aldehyde hydrate). In this approach, a primary amine of the hGH moiety reacts with the carbonyl group of the aldehyde or ketone (or the corresponding hydroxyl-containing group of a hydrated aldehyde or ketone), thereby forming a Schiff base. The Schiff base, in turn, can then be reduced to a stable conjugate through the use of a reducing agent such as sodium borohydride, sodium cyanoborohydride, or the like. Selective reactions (e.g., at the N-terminus) are possible, particularly with a polymer functionalized with a ketone or an alpha-methyl branched aldehyde and/or under specific reaction conditions (e.g., reduced pH). Representative synthetic methods for conjugating an aldehyde or ketone-functionalized polymeric reagent to an hGH moiety are described in Examples 3, 4 and 5.

Thus, one preferred conjugate in accordance with the invention, i.e., one having a water-soluble polymer covalently attached to an hGH moiety via a secondary amine linkage, comprises the following structure: 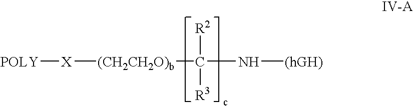
where POLY is a water-soluble polymer; X is a spacer moiety comprised of one or more atoms; (b) is an integer having a value of one through 10; (c) is an integer having a value of one through 10; R², in each occurrence, is independently H or an organic radical; R³, in each occurrence, is independently H or an organic radical; and hGH is a residue of a human growth hormone moiety, with the proviso that if POLY is branched, POLY does not contain a lysine residue. In one or more particular embodiments, X comprises from 1 to about 50 atoms, preferably from about 1 to about 25 atoms. In one or more particular embodiments, X comprises —C(O)NH—, or is —C(O)NH—.

Yet another exemplary conjugate possesses the following structure: 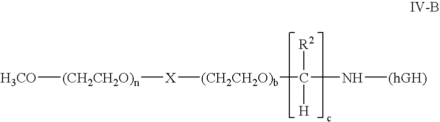
where (n) is an integer having a value of from 3 to 4000, preferably from 150 to 2200; X is as previously defined; (b) is an integer having a value of from 1 to 10, or preferably from 2 through 6; (c) is an integer having a value of from 1 to 10, preferably from 2 through 6; R², in each occurrence, is independently H or lower alkyl; and hGH is a residue of a human growth hormone moiety.

In yet a more specific embodiment of the generalized structure provided above, a preferred conjugate of the invention comprises the following structure: 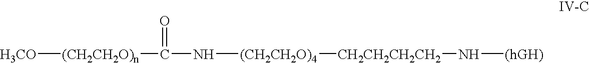
wherein (n) is as described above. The spacer moiety, X, in the structure above corresponds to —C(O)NH—, while R in each occurrence is H.

In another embodiment where POLY is a branched polymer, an illustrative conjugate possesses the following structure: 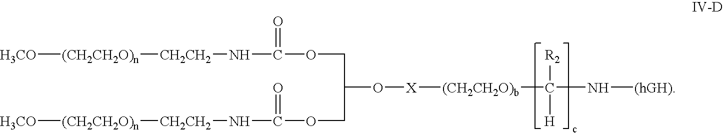
In the immediately preceding structure, each (n) is independently an integer having a value of from 3 to 4000, preferably from 150 to 2200, X is as previously defined; (b) is an integer having a value from 1 to 10, more preferably from 2 through 6; (c) is an integer having a value from 1 to 10, preferably from 2 through 6; R², in each occurrence, is independently H or lower alkyl; and hGH is a residue of human growth hormone.

In yet an even more particular embodiment, an exemplary conjugate of the invention comprises the following structure: 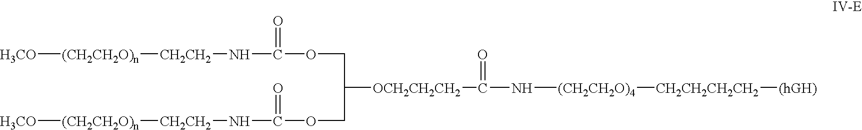
In the above structure, each (n) is independently an integer having a value of from 3 to 4000, more preferably from 150 to 2200; and hGH is a residue of human growth hormone.

Additional exemplary conjugates of the invention wherein the water-soluble polymer is in a branched form, may also comprise the following structure for the water-soluble polymer: 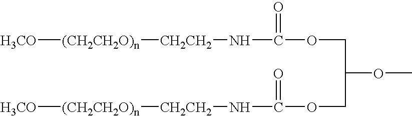
wherein each (n) is independently an integer having a value of from 3 to 4000, or preferably from 150 to 2200.

Carboxyl groups represent another functional group that can serve as a point of attachment on the hGH moiety. Structurally, the conjugate will comprise the following: 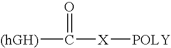
where (hGH) and the adjacent carbonyl group corresponds to the carboxyl-containing hGH moiety, X is a linkage, preferably a heteroatom selected from O, N(H), and S, and POLY is a water-soluble polymer such as PEG, optionally terminating in an end-capping moiety.

The C(O)—X linkage results from the reaction between a polymeric reagent bearing a terminal functional group suitable for reacting with a carboxyl group, and a carboxyl-containing hGH moiety. As discussed above, the specific linkage will depend on the type of functional group utilized. If the polymer is end-functionalized or “activated” with a hydroxyl group, then the resulting linkage will be a carboxylic acid ester, and X will be O. If the polymer is functionalized with a thiol group, the resulting linkage will be a thioester, and X will be S. When certain multi-arm, branched or forked polymers are employed, the C(O)X moiety, and in particular the X moiety, may be relatively more complex and may include a longer linkage structure.

Water-soluble derivatives containing a hydrazide moiety are also useful for conjugation at a carbonyl. To the extent that the hGH moiety does not contain a carbonyl moiety, a carbonyl moiety can be introduced by reducing any carboxylic acid (e.g., the C-terminal carboxylic acid) and/or by providing glycosylated or glycated (wherein the added sugars have a carbonyl moiety) versions of the hGH moiety. In an alternative approach to introducing an aldehyde functionality into the hGH moiety, a hydroxy group within the protein can be oxidized to an aldehyde, which is then suitable for reaction with a polymeric hydrazine reagent such as those shown below. Specific examples of polymeric reagents containing a hydrazide moiety, along with the corresponding conjugates, are provided in Table 2 below. In addition, any water-soluble polymer derivative containing an activated ester (e.g., a succinimidyl group) can be converted to contain a hydrazide moiety by reacting the water-soluble polymer derivative containing the activated ester with hydrazine (NH₂—NH₂) or tert-butyl carbazate [NH₂NHCO₂C(CH₃)₃]. In the table, the variable (n) represents the number of repeating monomeric units and “(hGH)” represents the residue of the hGH moiety following conjugation to the polymeric reagent. Optionally, the resulting hydrazone linkage can be reduced using a suitable reducing agent such as sodium cyanoborohydride. While each polymeric portion [e.g., (OCH₂CH₂)ₙ or (CH₂CH₂O)ₙ] presented in Table 2 terminates in a “CH₃” group, other groups (such as H and benzyl) can be substituted therefor.

TABLE 2
Carboxyl-Specific Polymeric Reagents and the hGH Moiety Conjugate Formed Therefrom

| Polymeric Reagent | Corresponding Conjugate |
|---|---|
| 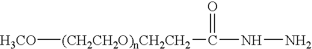 | 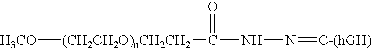 |
| mPEG-Hydrazine Derivative | Hydrazone Linkage |
| 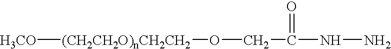 | 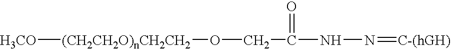 |
| mPEG-Hydrazine Derivative | Hydrazone Linkage |
| 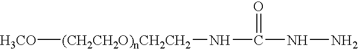 | 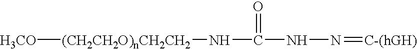 |
| mPEG-Hydrazine Derivative | Hydrazone Linkage |
| 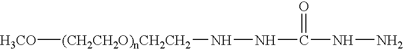 | 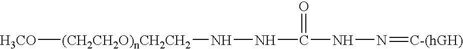 |
| mPEG-Hydrazine Derivative | Hydrazone Linkage |
| 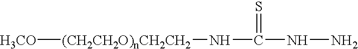 | 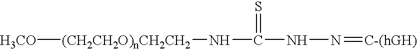 |
| mPEG-Hydrazine Derivative | Hydrazone Linkage |
| 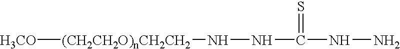 | 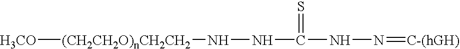 |
| mPEG-Hydrazine Derivative | Hydrazone Linkage |
| 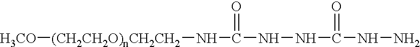 | 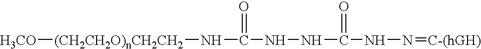 |
| mPEG-Hydrazine Derivative | Hydrazone Linkage |
| 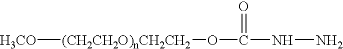 | 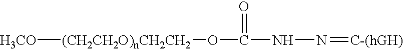 |
| mPEG-Hydrazine Derivative | Hydrazone Linkage |

Thiol groups contained within the hGH moiety can also serve as effective sites of attachment for the water-soluble polymer. In particular, cysteine residues provide thiol groups when the hGH moiety is a protein. The thiol groups in such cysteine residues can then be reacted with an activated PEG that is specific for reaction with thiol groups, e.g., an N-maleimidyl polymer or other derivative, as described in U.S. Pat. No. 5,739,208 and in International Patent Publication No. WO 01/62827.

With respect to SEQ ID NOs: 1 through 4, there are four thiol-containing cysteine residues. Thus, preferred thiol attachment sites are associated with one of these four cysteine residues. Although it is preferred not to disrupt any disulfide bonds, it may be possible to attach a polymer within the side chain of one or more of these cysteine residues and retain a degree of activity. To the extent that any particular hGH moiety lacks a thiol group or disruption of disulfide bonds is to be avoided, however, it is possible to add a cysteine residue to the hGH moiety using conventional synthetic techniques. See, for example, WO 90/12874. In addition, conventional genetic engineering processes can also be used to introduce a cysteine residue into the hGH moiety. In some embodiments, however, it is preferred not to introduce and additional cysteine residue and/or thiol group.

Specific examples of conjugates resulting from reaction of a suitably functionalized polymeric reagent with a thiol group of an hGH moiety are provided below. Useful water-soluble polymers are provided in the left-hand column of Table 3 below, along with the corresponding conjugate in the right-hand column. In the table, the variable (n) represents the number of repeating monomeric units and “(hGH)” represents the hGH moiety residue following conjugation to the water-soluble polymer via a thiol-group. While each polymeric portion [e.g., (OCH₂CH₂)ₙ or (CH₂CH₂O)ₙ] presented in Table 3 terminates in a “CH₃” group, other groups (such as H and benzyl) can be substituted therefor.

TABLE 3
Thiol-Specific Polymeric Reagents and the hGH Moiety Conjugate Formed Therefrom
Polymeric Reagent

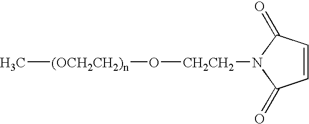

mPEG Maleimide Derivative

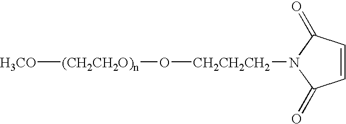

mPEG Maleimide Derivative

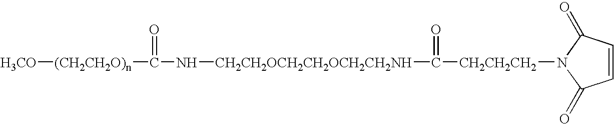

mPEG Maleimide Derivative

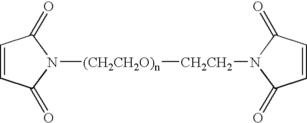

Homobifunctional mPEG Maleimide
Derivative

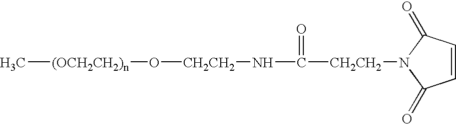

mPEG Maleimide Derivative

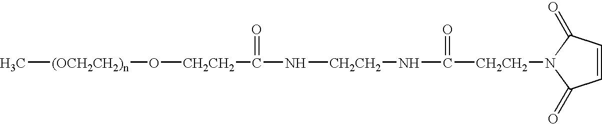

mPEG Maleimide Derivative

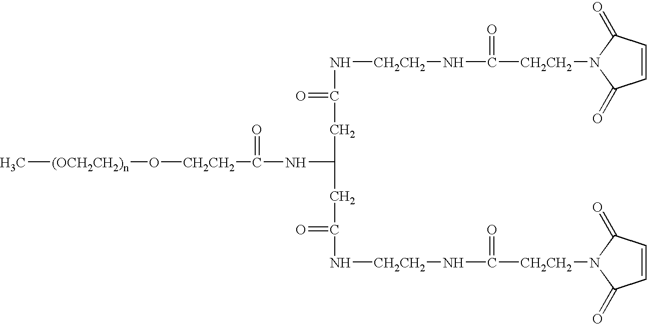

mPEG Forked Maleimide Derivative

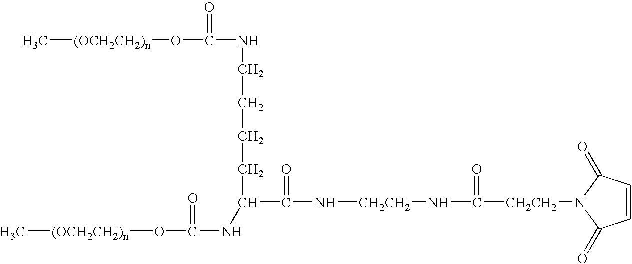

branched mPEG2 Maleimide Derivative

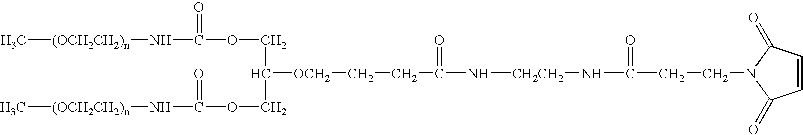

branched mPEG2 Maleimide Derivative

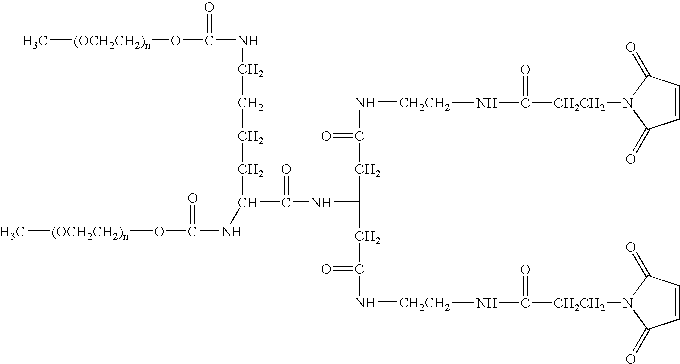

Branched mPEG2 Forked Maleimide
Derivative

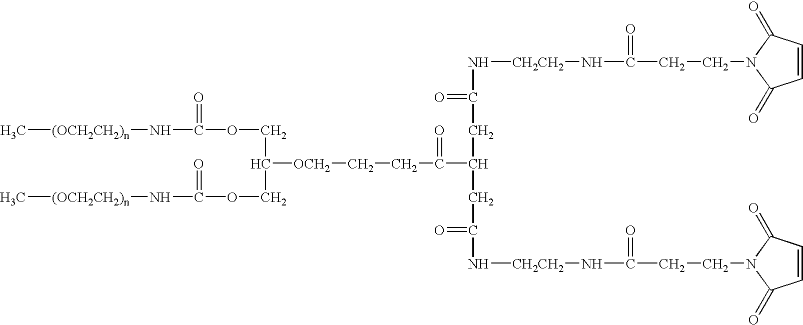

Branched mPEG2 Forked Maleimide
Derivative

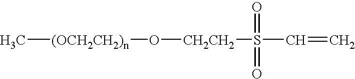

mPEG Vinyl Sulfone Derivative

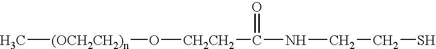

mPEG Thiol Derivative

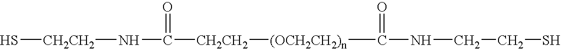

Homobifunctional PEG Thiol Derivative

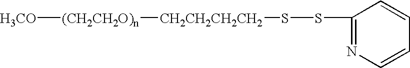

mPEG Disulfide Derivative
As described in copending U.S. Provisional
Application No. 60/639,823 filed on
Dec. 21, 2004 and entitled “Stabilized Polymeric
Thiol Reagents.”)

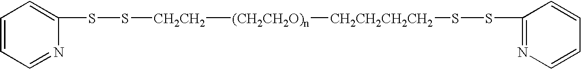

Homobifunctional Disulfide Derivative
As described in copending U.S. Provisional
Application No. 60/639,823 filed on
Dec. 21, 2004 and entitled “Stabilized Polymeric
Thiol Reagents.”)
Thiol-Specific Polymeric Reagents and the hGH Moiety Conjugate Formed Therefrom
Corresponding Conjugate

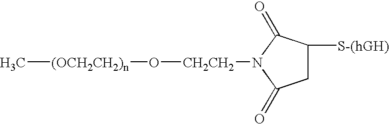

Thioether Linkage

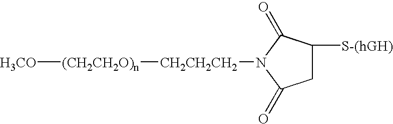

Thioether Linkage

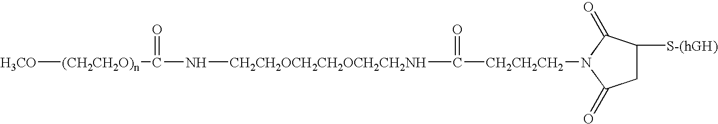

Thioether Linkage

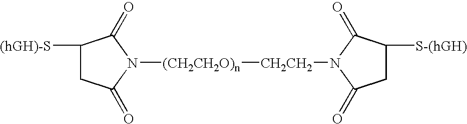

Thioether Linkages

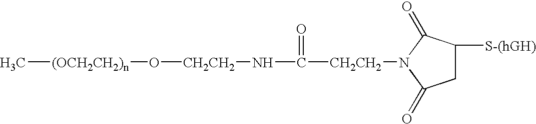

Thioether Linkage

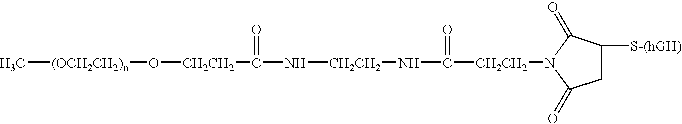

Thioether Linkage

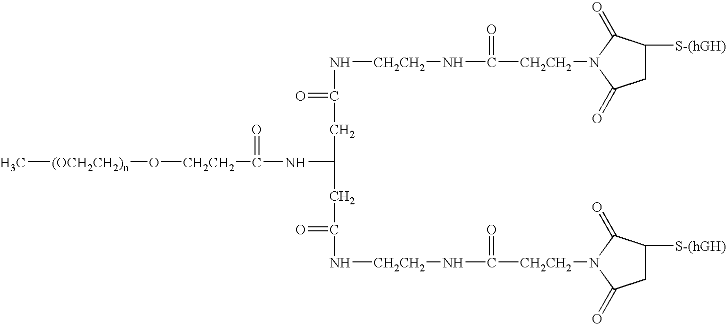

Thioether Linkage

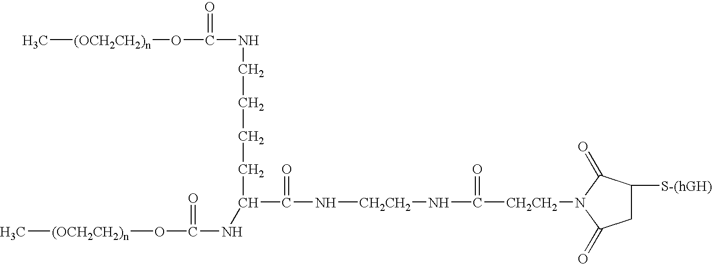

Thioether Linkage

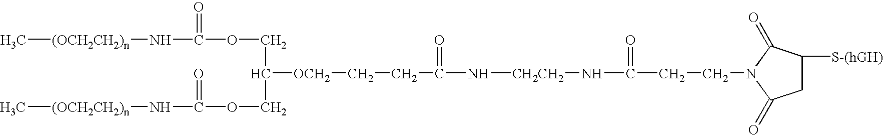

Thioether Linkage

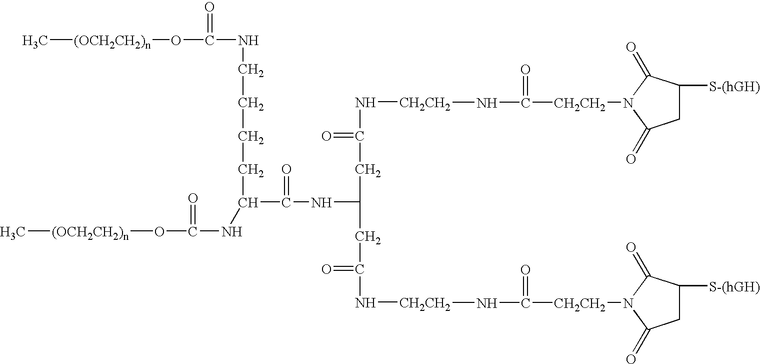

Thioether Linkages

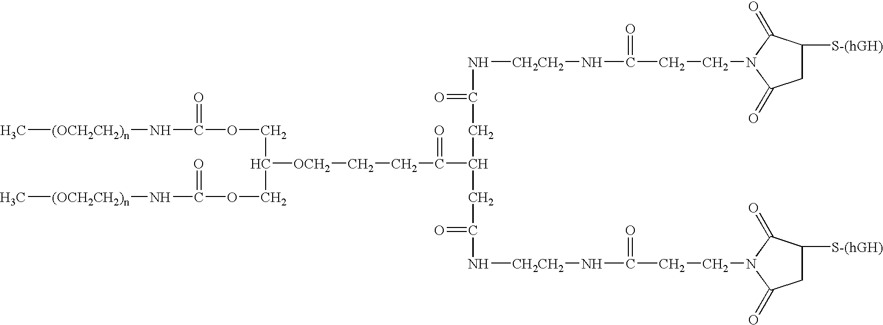

Thioether Linkages

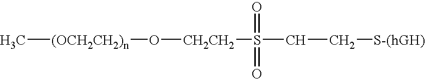

Thioether Linkage

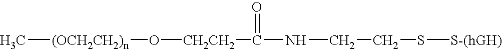

Disulfide Linkage

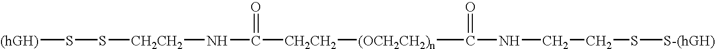

Disulfide Linkages

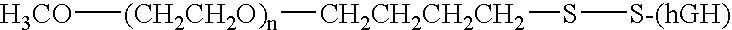

Disulfide Linkage

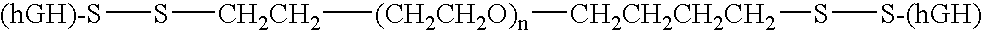

Disulfide Linkages

With respect to conjugates formed from water-soluble polymers bearing one or more maleimide functional groups (regardless of whether the maleimide reacts with an amine or thiol group on the hGH moiety), the corresponding maleamic acid form(s) of the water-soluble polymer can also react with the hGH moiety. Under certain conditions (e.g., a pH of about 7-9 and in the presence of water), the maleimide ring will “open” to form the corresponding maleamic acid. The maleamic acid, in turn, can react with an amine or thiol group of an hGH moiety. Exemplary maleamic acid-based reactions are schematically shown below. POLY represents the water-soluble polymer, and (hGH) represents the hGH moiety. 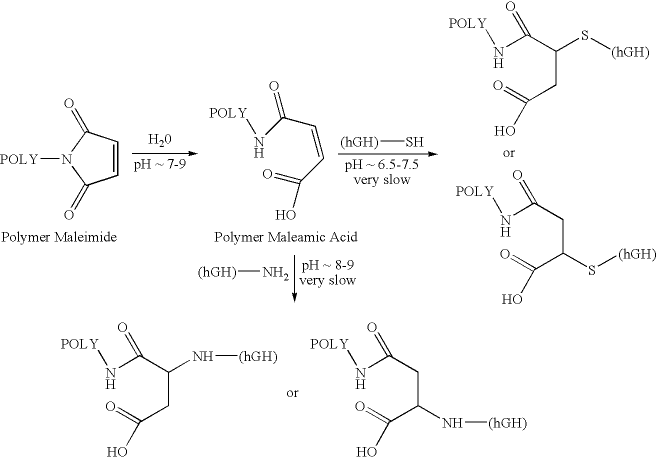
A representative conjugate in accordance with the invention may therefore possess the following structure:
POLY-L_{0,1}-C(O)Z-Y—S—S-(hGH)
wherein POLY is a water-soluble polymer, L is an optional linker, Z is a heteroatom selected from the group consisting of O, NH, and S, and Y is selected from the group consisting of C₂₋₁₀ alkyl, C₂₋₁₀ substituted alkyl, aryl, and substituted aryl, and (hGH) is an hGH moiety. Polymeric reagents that can be reacted with an hGH moiety and result in this type of conjugate are described in U.S. Patent Application Publication No. 2005/0014903.

Preferred thiol groups in an hGH moiety that can serve as a site for attaching a polymeric reagent include those thiol groups found within cysteine residues.

With respect to polymeric reagents, those described here and elsewhere can be purchased from commercial sources (e.g., Nektar Therapeutics, Huntsville, Ala.). In addition, methods for preparing such polymeric reagents are described in the literature, or can be readily determined by one skilled in the chemical synthetic arts.

The attachment between the hGH moiety and the non-peptidic water-soluble polymer can be direct, wherein no intervening atoms are located between the hGH moiety and the polymer, or indirect, wherein one or more atoms are located between the hGH moiety and the polymer. With respect to the indirect attachment, the one or more atoms is conventionally referred to as a “spacer moiety,” which can include one or more of carbon atoms, nitrogen atoms, sulfur atoms, oxygen atoms, and combinations thereof. The spacer moiety can comprise an amide, secondary amine, carbamate, thioether, or disulfide group. Nonlimiting examples of specific spacer moieties include those selected from the group consisting of —O—, —S—, —S—S—, —C(O)—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—[CH₂]ₕ—(OCH₂CH₂)ⱼ—, bivalent cycloalkyl group, —O—, —S—, an amino acid, —N(R⁶)—, and combinations of two or more of any of the foregoing, wherein R⁶ is H or an organic radical selected from the group consisting of alkyl, substituted alkyl, alkenyl, substituted alkenyl, alkynyl, substituted alkynyl, aryl and substituted aryl, (h) is zero to six, and (j) is zero to 20. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁₋₆—NH—C(O)—, wherein the subscript values following each methylene indicate the number of methylenes contained in the structure, e.g., (CH₂)₁-6 means that the structure can contain 1, 2, 3, 4, 5 or 6 methylenes. Additionally, any of the above spacer moieties may further include an ethylene oxide oligomer chain comprising 1 to 20 ethylene oxide monomer units [i.e., —(CH₂CH₂O)₁₋₂₀]. That is, the ethylene oxide oligomer chain can occur before or after the spacer moiety, and optionally in between any two atoms of a spacer moiety comprised of two or more atoms. Also, the oligomer chain would not be considered part of the spacer moiety if the oligomer is adjacent to a polymer segment and merely represent an extension of the polymer segment.

Compositions

The hGH conjugates provided herein are typically part of a composition. Generally, the composition comprises a plurality of conjugates, preferably although not necessarily, each conjugate is comprised of the same hGH moiety (i.e., within the entire composition, only one type of hGH moiety is found). In addition, the composition can comprise a plurality of conjugates wherein any given conjugate is comprised of a moiety selected from the group consisting of two or more different hGH moieties (i.e., within the entire composition, two or more different hGH moieties are found). Optimally, however, substantially all conjugates in the composition (e.g., 85% or more of the plurality of conjugates in the composition) are each comprised of the same hGH moiety.

The composition can comprise a single conjugate species (e.g., a monoPEGylated conjugate wherein the single polymer is attached at the same location for substantially all conjugates in the composition) or a mixture of conjugate species (e.g., a mixture of monoPEGylated conjugates where attachment of the polymer occurs at different sites and/or is a mixture monoPEGylated, diPEGylated and triPEGylated conjugates). The compositions can also comprise other conjugates having four, five, six, seven, eight or more polymers attached to any given moiety having hGH activity. In addition, the invention includes instances wherein the composition comprises a plurality of conjugates, each conjugate comprising one water-soluble polymer covalently attached to one hGH moiety, as well as compositions comprising two, three, four, five, six, seven, eight, or more water-soluble polymers covalently attached to one hGH moiety. In one preferred embodiment, the composition is substantially monoPEGylated conjugate, where 80% or more of the PEGylated species comprising the composition, or more preferably 90% or more, is monoPEGylated. In yet another preferred embodiment, a composition of the invention will comprise substantially diPEGylated species, where 80% or more of the PEGylated species comprising the composition is diPEGylated, or more preferably 85% or more of the PEGylated species comprising the composition is diPEGylated.

With respect to the conjugates in the composition, the composition will satisfy one or more of the following characteristics: at least about 85% of the conjugates in the composition will have from one to four polymers attached to the hGH moiety; at least about 85% of the conjugates in the composition will have from one to three polymers attached to the hGH moiety; at least about 85% of the conjugates in the composition will have from one to two polymers attached to the hGH moiety; at least about 85% of the conjugates in the composition will have one polymer attached to the hGH moiety; at least about 95% of the conjugates in the composition will have from one to four polymers attached to the hGH moiety; at least about 95% of the conjugates in the composition will have from one to three polymers attached to the hGH moiety; at least about 95% of the conjugates in the composition will have from one to two polymers attached to the hGH moiety; at least about 95% of the conjugates in the composition will have one polymer attached to the hGH moiety; at least about 99% of the conjugates in the composition will have from one to four polymers attached to the hGH moiety; at least about 99% of the conjugates in the composition will have from one to three polymers attached to the hGH moiety; at least about 99% of the conjugates in the composition will have from one to two polymers attached to the hGH moiety; and at least about 99% of the conjugates in the composition will have one polymer attached to the hGH moiety.

In one or more embodiments, it is preferred that the conjugate-containing composition is free or substantially free of albumin. It is also preferred that the composition is free or substantially free of proteins that do not have hGH activity. Thus, it is preferred that the composition is 85%, more preferably 95%, and most preferably 99% free of albumin. Additionally, it is preferred that the composition is 85%, more preferably 95%, and most preferably 99% free of any protein that does not have hGH activity.

Control of the desired number of polymers for any given moiety can be achieved by selecting the proper polymeric reagent, the ratio of polymeric reagent to the hGH moiety, temperature, pH conditions, and other aspects of the conjugation reaction. In addition, reduction or elimination of the undesired conjugates (e.g., those conjugates having four or more attached polymers) can be achieved through purification means.

For example, the polymer-hGH moiety conjugates can be purified to obtain/isolate different conjugated species. Specifically, the product mixture can be purified to obtain an average of anywhere from one, two, three, four, five or more PEGs per hGH moiety, typically one, two or three PEGs per hGH moiety. Preferred are one or two PEGs per hGH moiety. The strategy for purification of the final conjugate reaction mixture will depend upon a number of factors, including, for example, the molecular weight of the polymeric reagent employed, the particular hGH moiety, the desired dosing regimen, and the residual activity and in vivo properties of the individual conjugate(s).

If desired, conjugates having different molecular weights can be isolated using gel filtration chromatography, reverse phase chromatography, hydrophobic interaction chromatography (HIC), and/or ion exchange chromatography. That is to say, gel filtration chromatography is used to fractionate differently numbered polymer-to-hGH moiety ratios (e.g., 1-mer, 2-mer, 3-mer, and so forth, wherein “1-mer” indicates 1 polymer to hGH moiety, “2-mer” indicates two polymers to hGH moiety, and so on) on the basis of their differing molecular weights (where the difference corresponds essentially to the average molecular weight of the water-soluble polymer portion). For example, in an exemplary reaction where a 35,000 Dalton protein is randomly conjugated to a polymeric reagent having a molecular weight of about 20,000 Daltons, the resulting reaction mixture may contain unmodified protein (having a molecular weight of about 35,000 Daltons), monoPEGylated protein (having a molecular weight of about 55,000 Daltons), diPEGylated protein (having a molecular weight of about 75,000 Daltons), and so forth.

While this approach can be used to separate PEG and other polymer-hGH moiety conjugates having different molecular weights, this approach is generally ineffective for separating positional isoforms having different polymer attachment sites within the hGH moiety. For example, gel filtration chromatography can be used to separate from each other mixtures of PEG 1-mers, 2-mers, 3-mers, and so forth, although each of the recovered conjugate compositions may contain PEG(s) attached to different reactive groups (e.g., lysine residues) within the hGH moiety.

Gel filtration columns suitable for carrying out this type of separation include Superdex™ and Sephadex™ columns available from Amersham Biosciences (Piscataway, N.J.). Selection of a particular column will depend upon the desired fractionation range desired. Elution is generally carried out using a suitable buffer, such as phosphate, acetate, or the like. The collected fractions may be analyzed by a number of different methods, for example, (i) absorbance at 280 nm for protein content, (ii) dye-based protein analysis using bovine serum albumin (BSA) as a standard, (iii) iodine testing for PEG content (Sims et al. (1980) Anal. Biochem, 107:60-63), (iv) sodium dodecyl sulfate polyacrylamide gel electrophoresis (SDS-PAGE), followed by staining with barium iodide, and (v) high performance liquid chromatography (HPLC).

Separation of positional isoforms is carried out by reverse phase chromatography using a reverse phase-high performance liquid chromatography (RP-HPLC) using a suitable column (e.g., a C18 column or C3 column, available commercially from companies such as Amersham Biosciences or Vydac) or by ion exchange chromatography using an ion exchange column, e.g., a Sepharose™ ion exchange column available from Amersham Biosciences. Either approach can be used to separate polymer-active agent isomers having the same molecular weight (i.e., positional isoforms).

Representative hGH conjugate-comprising compositions of the invention comprise the following: 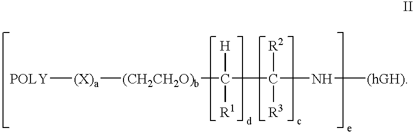
In structure II, POLY is a water-soluble polymer; (a) is either zero or one; (b) is either zero or an integer having a value from 1 to 10; (c) is an integer having a value from 1 to 10; (d) is either zero or one; (e) is either one or two; X, when present, is a spacer moiety comprised of one or more atoms. R¹ is H or an organic radical containing from 1 to 3 carbon atoms. Either R² and R³, when taken together with the carbon atom to which they are attached, in each occurrence considered separately, forms a carbonyl (C═O), or R², in each occurrence, is independently H or an organic radical, and R³, in each occurrence, is independently H or an organic radical. hGH is a residue of a human growth hormone moiety.

In one or more embodiments of the invention, one or more of the following provisos (i) to (iii) may apply to compositions comprising structure II above: (i) when R² and R³, when taken together with the carbon atom to which they are attached form a carbonyl adjacent to the —NH—, and POLY is linear, then (d) equals one and R¹ is an organic radical containing from 1 to 3 carbon atoms; (ii) when R² and R³, in each occurrence, are each independently H or an organic radical, then (b) is a positive integer and (a) is one; and (iii) when POLY is branched, POLY does not contain a lysine residue. In certain instances, only proviso (i) will apply. In other instances, only proviso (ii) will apply. In other instances, only proviso (iii) will apply. In yet other instances, certain combinations of provisos will apply, e.g., (i) and (ii), (i) and (iii), and (ii) and (iii). In yet one or more additional embodiments, all of the foregoing provisos (i)-(iii) apply to structure II above.

In a particular embodiment, provided is a composition comprising a polymer of structure II above where each of provisos (i)-(iii) applies, and further wherein when (e) is one, the composition comprises 85% or greater monoconjugate with respect to all conjugate species contained in the composition, and when (e) is two, the composition comprises greater than 80% diconjugate with respect to all conjugate species contained in the composition, and said composition is bioactive.

Preferably, in reference to structure II, the acyclic spacer moiety, X, comprises from 1 to about 50 atoms, or even more preferably, from about 1 to about 25 atoms.

In yet one or more additional embodiments of structure II, R² and R³, when taken together with the carbon atom to which they are attached, form a carbonyl adjacent to the —NH— and POLY is branched or linear. Preferably, POLY is branched. In yet another embodiment of structure II, (b) and (d) both equal zero, and (c) equals 4.

According to yet another embodiment in reference to structure II and compositions thereof, POLY is branched, (a) is one, (b) ranges from 1 to 10, (c) ranges from 1 to 6, and (d) is zero. In one such preferred embodiment, (b) is 4. In yet one or more additional embodiments, (c) is 4 and R² and R³ are both H in each occurrence. In yet one or more related embodiments, X comprises —CH₂)₃—C(O)—NH—. In yet another related embodiment, POLY comprises the structure: 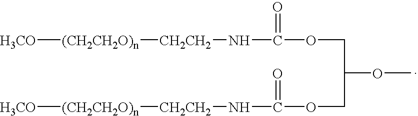
where each (n) is independently an integer having a value of from 150 to 2200.

According to yet one or more additional embodiments in reference to structure II and compositions thereof, POLY is linear, (a) is one, (b) ranges from 1 to 10, (c) ranges from 1-6, and (d) is zero. In a preferred embodiment thereof, (b) is 4. In yet another related embodiment, (c) is 4, and R² and R³ are both H in each occurrence. In yet another related embodiment thereof, X preferably comprises —C(O)—NH—. Even more particularly, in a preferred embodiment, POLY is H₃CO—(CH₂CH₂O)ₙ— and (n) is an integer having a value of from 150 to 2200.

In yet one or more additional embodiments, an hGH conjugate-comprising composition is one where the amount of monoconjugate and diconjugate in the composition represents at least about 85% of the total conjugate species in the composition. In a preferred embodiment, the amount of monoconjugate and diconjugate in the composition represents at least about 90% of the total conjugate species in the composition, and in a most preferred embodiment, the amount of monoconjugate and diconjugate in the composition represents at least about 95% of the total conjugate species in the composition.

The compositions are preferably substantially free of proteins that do not have hGH activity. In addition, the compositions preferably are substantially free of all other noncovalently attached water-soluble polymers. In some circumstances, however, the composition can contain a mixture of polymer-hGH moiety conjugates and unconjugated hGH moiety.

Optionally, the composition of the invention further comprises a pharmaceutically acceptable excipient. If desired, the pharmaceutically acceptable excipient can be added to a conjugate to form a composition.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The composition can also include an antimicrobial agent for preventing or deterring microbial growth. Nonlimiting examples of antimicrobial agents suitable for one or more embodiments of the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the conjugate or other components of the preparation. Suitable antioxidants for use in one or more embodiments of the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the conjugate (i.e., the conjugate formed between the active agent and the polymeric reagent) in the composition will vary depending on a number of factors, but will optimally be a therapeutically effective dose when the composition is stored in a unit dose container (e.g., a vial). In addition, the pharmaceutical preparation can be housed in a syringe. A therapeutically effective dose can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15% to about 95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The compositions encompass all types of formulations and in particular those that are suited for injection, e.g., powders or lyophilates that can be reconstituted as well as liquids. Examples of suitable diluents for reconstituting solid compositions prior to injection include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

The compositions of one or more embodiments of the present invention are typically, although not necessarily, administered via injection and are therefore generally liquid solutions or suspensions immediately prior to administration. The pharmaceutical preparation can also take other forms such as syrups, creams, ointments, tablets, powders, and the like. Other modes of administration are also included, such as pulmonary, rectal, transdermal, transmucosal, oral, intrathecal, subcutaneous, intra-arterial, and so forth.

Administration

The invention also provides a method for administering a conjugate as provided herein to a patient suffering from a condition that is responsive to treatment with the hGH moiety employed. The method comprises administering to a patient, generally via injection, a therapeutically effective amount of the conjugate (preferably provided as part of a pharmaceutical composition). As previously described, the conjugates can be administered parenterally by intravenous injection, or less preferably by intramuscular or by subcutaneous injection. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others.

The method of administering may be used to treat any condition that can be remedied or prevented by administration of the hGH conjugate. Those of ordinary skill in the art appreciate which conditions a specific conjugate can effectively treat. For example, the conjugates of the invention can be used to treat patients suffering from acromegaly, diabetic retinopathy, an inadequate secretion of endogenous growth hormone (dwarfism), Prader-Willi syndrome (PWS), cachexia, Turner's syndrome, long term treatment of children who are born short for gestational age, hypopituitarism, somatotropin deficiency, renal insufficiency, AIDS wasting, end stage renal failure, and cystic fibrosis. Additionally, the conjugates and compositions of the invention may be used as part of adult hGH replacement therapy. The aim of human growth hormone replacement therapy is typically to restore hGH levels in a subject to those levels typically seen in early adulthood. In a healthy 21 year old, normal levels of circulating hGH are 10 mg per deciliter of blood; in a 61 year-old, the average level of circulating hGH is 2 mg/dl. When used as part of a replacement therapy regimen, an hGH conjugate of the invention may be administered in combination with DHEA, or with estrogen/progesterone when used in treatment of a female subject, or with testosterone when used in treatment of a male subject.

Advantageously, the conjugate can be administered to the patient prior to, simultaneously with, or after administration of another active agent.

The actual dose to be administered will vary depending upon the age, weight, and general condition of the subject as well as the severity of the condition being treated, the judgment of the health care professional, and conjugate being administered. Therapeutically effective amounts are known to those skilled in the art and/or are described in the pertinent reference texts and literature. Generally, a therapeutically effective amount will range from about 0.001 mg to 100 mg, preferably in doses from 0.01 mg/day to 75 mg/day, and more preferably in doses from 0.10 mg/day to 50 mg/day. A given dose can be periodically administered up until, for example, a target height is achieved or epiphyseal fusion occurs.

The unit dosage of any given conjugate (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration once daily, three times weekly, twice weekly, once weekly, twice monthly, once monthly, and any combination thereof. Once the clinical endpoint has been achieved, dosing of the composition is halted.

One advantage of administering certain conjugates described herein is that individual water-soluble polymer portions can be cleaved. Such a result is advantageous when clearance from the body is potentially a problem because of the polymer size. Optimally, cleavage of each water-soluble polymer portion is facilitated through the use of physiologically cleavable and/or enzymatically degradable linkages such as amide, carbonate or ester-containing linkages. In this way, clearance of the conjugate (via cleavage of individual water-soluble polymer portions) can be modulated by selecting the polymer molecular size and the type of functional group that would provide the desired clearance properties. One of ordinary skill in the art can determine the proper molecular size of the polymer as well as the cleavable functional group. For example, one of ordinary skill in the art, using routine experimentation, can determine a proper molecular size and cleavable functional group by first preparing a variety of polymer derivatives with different polymer weights and cleavable functional groups, and then obtaining the clearance profile (e.g., through periodic blood or urine sampling) by administering the polymer derivative to a patient and taking periodic blood and/or urine sampling. Once a series of clearance profiles have been obtained for each tested conjugate, a suitable conjugate can be identified.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All articles, books, patents and other publications referenced herein are hereby incorporated by reference in their entireties.

### EXPERIMENTAL

The practice of the invention will employ, unless otherwise indicated, conventional techniques of organic synthesis, biochemistry, protein purification and the like, which are within the skill of the art. Such techniques are fully explained in the literature. See, for example, J. March, Advanced Organic Chemistry: Reactions Mechanisms and Structure, 4th Ed. (New York: Wiley-Interscience, 1992), supra.

In the following examples, efforts have been made to ensure accuracy with respect to numbers used (e.g., amounts, temperatures, etc.) but some experimental error and deviation should be accounted for. Unless indicated otherwise, temperature is in degrees C. and pressure is at or near atmospheric pressure at sea level. Each of the following examples is considered to be instructive to one of ordinary skill in the art for carrying out one or more of the embodiments described herein.

rhGH corresponding to the amino acid sequence of SEQ. ID. NO. 1. was used in Examples 1-4. The rhGH stock solution contained sodium phosphate buffer (ph 7.2) at a concentration of 7 mg/mL. Conjugation reactions were quenched with Tris [tris(hydroxy methyl) amino methane].

SDS-PAGE Analysis

Samples are analyzed by sodium dodecyl sulfate-polyacrylamide gel electrophoresis (SDS-PAGE) using Bio-Rad system (Mini-PROTEAN III Precast Gel Electrophoresis System). Samples are mixed with sample buffer. Then, the prepared samples are loaded onto a gel and run for approximately thirty minutes.

Anion Exchange Chromatography

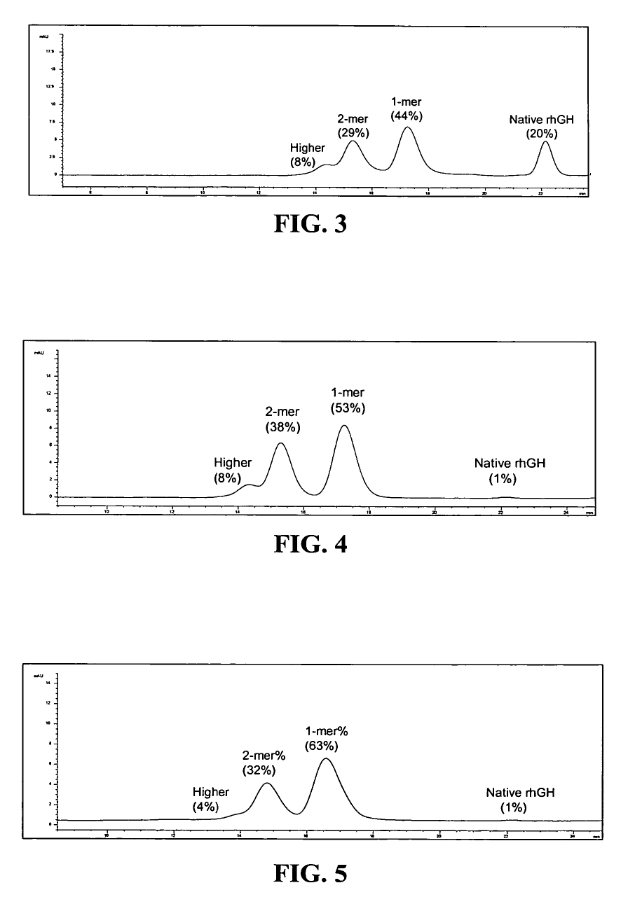

A Hitrap Q Sepharose FF anion exchange column (5 ml, Amersham Biosciences) was used with the AKTAprime system (Amersham Biosciences) to purify the PEG-rhGH conjugates prepared in Example 1. For each conjugate solution prepared, the conjugate solution was loaded on a column that was pre-equilibrated in 20 mM Tris buffer, pH 7.5 (buffer A) and then washed with nine column volumes of buffer A to remove any unreacted PEG reagent. Subsequently, a gradient of buffer A with 0-100% buffer B (20 mM Tris with 0.5 M NaCl buffer, pH 7.5) was raised. The eluent was monitored by UV detector at 280 nm. Any higher-mers (e.g., 3-mers, 4-mers, and so forth) and 2-mers were eluted first, followed by 1-mers, and finally the unconjugated rhGH. The fractions were pooled according to the chromatogram (FIG. 3), and the purity of the individual conjugate was determined by SEC-HPLC (FIGS. 4 and 5).

SEC-HPLC Analysis

Size exclusion chromatography (SEC-HPLC) analysis was performed on an Agilent 1100 HPLC system (Agilent). Samples were analyzed using a Shodex protein KW-804 column (300×8 mm, Phenomenex), and a mobile phase consisting of 90% phosphate buffered saline and 10% ethanol, pH 7.4. The flow rate for the column was 0.5 ml/min. Eluted protein and PEG-protein conjugates were detected using UV at 280 nm.

### Example 1

### PEGylation of rhGH with Branched mPEG-N-Hydroxysuccinimide Derivative, 40 kDa

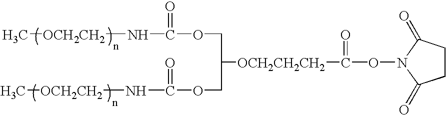

### Branched mPEG-N-Hydroxysuccinimide Derivative, 40 kDa, (“mPEG-2-NHS”)

mPEG-2-NHS, 40 kDa, stored at −20° C. under argon, was warmed to ambient temperature. A five-fold excess (relative to the amount of rhGH in a measured aliquot of the stock rhGH solution) of the warmed mPEG-2-NHS was dissolved in 10 mM sodium phosphate (pH 4.9) to form a 10% reagent solution. The 10% reagent solution was quickly added to the aliquot of stock rhGH solution and mixed well. After the addition of the mPEG-2-NHS, the pH of the reaction mixture was determined and adjusted to 6.7 to 6.8 using conventional techniques. To allow for coupling of the mPEG-2-NHS to rhGH via an amide linkage, the reaction solution was stirred for five hours at room temperature in the dark, thereby resulting in a conjugate solution. The reaction was quenched with Tris buffer. The conjugate solution was characterized.

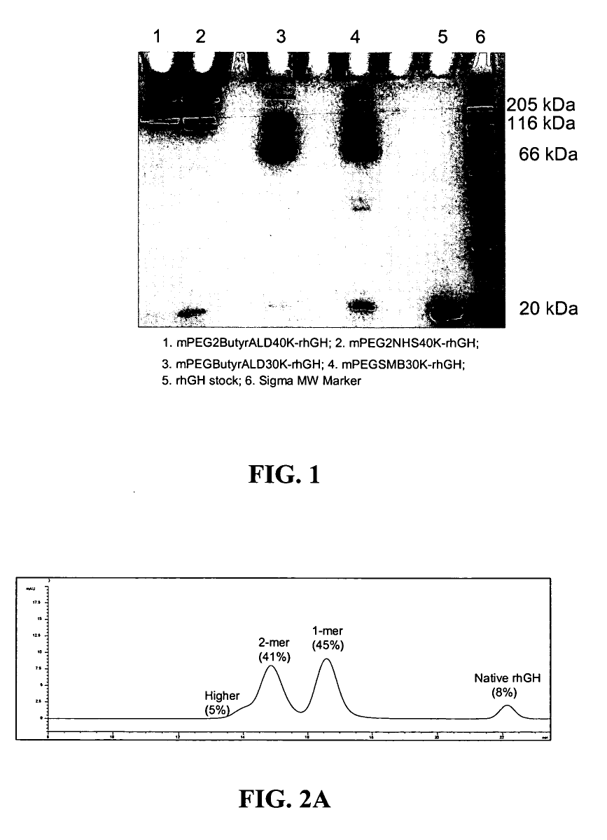

Lane 2 in FIG. 1 shows the SDS-PAGE analysis of this conjugate solution.

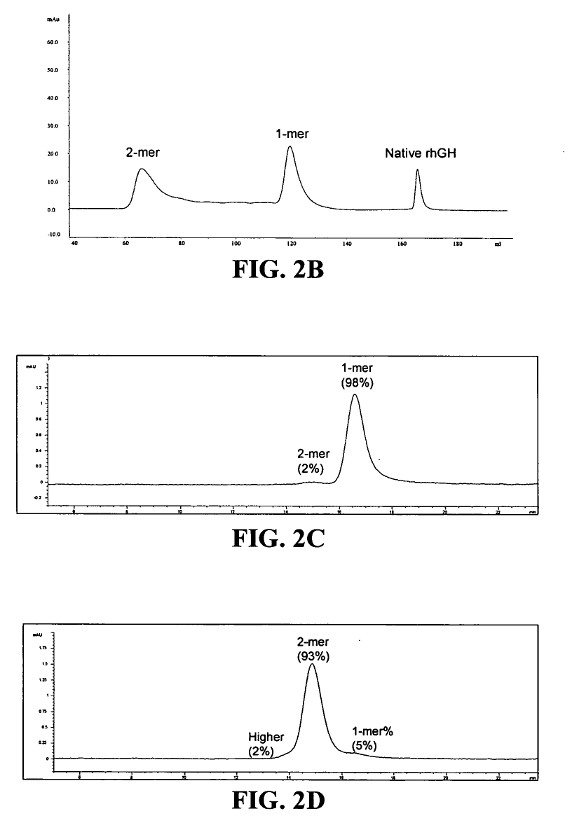

FIG. 2A shows the chromatogram following the SEC-HPLC analysis of the conjugate reaction mixture. The PEGylation reaction yielded 45% 1-mer (one PEG attached to rhGH or monoPEGylated) and 41% 2-mer (two PEGs attached to rhGH or diPEGylated) species. The total percentages in FIG. 2A do not add to 100% due to rounding.

Anion-exchange chromatography was used to purify the conjugates. FIG. 2B shows the chromatogram following anion-exchange purification. The conjugate fractions were collected and analyzed by SEC-HPLC. The purified 1-mer, mono(mPEG-2-NHS-40 k) hGH, (MW: 65 kDa, per MALDI-TOF), was shown to be 98% pure (FIG. 2C), while the purified 2-mer, di(mPEG-2-NHS-40 k) hGH (MW: 108 kDa, per MALDI-TOF), was shown to be 93% pure (FIG. 2D).

mPEG-2-NHS provided a relatively large molecular volume of active N-hydroxysuccinimide (“NHS”) ester, which selectively reacted with lysine and terminal amines. This is because the PEGylation reaction was carried out at pH 6.7 to 6.8, which favors N-terminal directed modification in conjunction with internal lysine modification.

Using this same approach, other conjugates can be prepared using mPEG-2-NHS having other weight average molecular weights.

### Example 2

### PEGylation of rhGH with Linear mPEG-Succinimidyl α-Methylbutanoate Derivative, 30 kDa

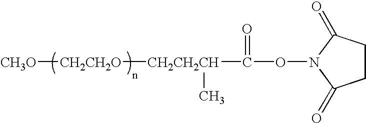

### Linear m-PEG-Succinimidyl α-Methylbutanoate Derivative, 30 kDa (“mPEG-SMB”)

mPEG-SMB, 30 kDa, stored at −20° C. under argon, was warmed to ambient temperature. A five-fold excess (relative to the amount of rhGH in a measured aliquot of the stock rhGH solution) of the warmed mPEG-SMB was dissolved in 10 mM sodium phosphate (pH 4.9) to form a 10% reagent solution. The 10% reagent solution was quickly added to the aliquot of stock rhGH solution and mixed well. After the addition of the mPEG-SMB, the pH of the reaction mixture was determined and adjusted to 6.7 to 6.8 using conventional techniques. To allow for coupling of the mPEG-SMB to rhGH via an amide linkage, the reaction solution was stirred for five hours at room temperature in the dark and thereafter stirred overnight at 3-8° C. in a cold room in the dark, thereby resulting in a conjugate solution. The reaction was quenched with Tris buffer. The conjugate solution was characterized.

Lane 4 in FIG. 1 shows the SDS-PAGE analysis of this conjugate solution.

The mPEG-SMB derivative provided a sterically hindered active NHS ester, which selectively reacted with lysine and terminal amines. Since the PEGylation reaction was carried out at pH 6.7 to pH 6.8, such reaction conditions favored N-terminal directed modification in conjunction with internal lysine modification. FIG. 3 shows the SEC-HPLC chromatogram of the conjugate reaction mixture. The SEC-HPLC analysis reveals the PEGylation reaction yielded 44% 1-mer (one PEG attached to rhGH or monoPEGylated), mono(mPEG-SMB-30 k) hGH, and 29% 2-mer (two PEGs attached to rhGH or diPEGylated) species, di(mPEG-SMB-30 k) hGH. An anion-exchange chromatography method using Q Sepharose Fast Flow and Tris buffer was also used to purify the conjugates. The profile of the purified conjugates was similar to that shown in FIG. 2B. The purified conjugates were up to 95% pure.

Using this same approach, other conjugates can be prepared using mPEG-SMB having other weight average molecular weights.

### Example 3

### PEGylation of rhGH with Linear mPEG-Butyraldehyde Derivative, 30 kDa

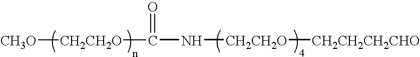

### Linear mPEG-Butyraldehyde Derivative, 30 kDa (“mPEG-ButyrALD”)

mPEG-ButyrALD, 30 kDa, stored at −20° C. under argon, was warmed to ambient temperature. An eight-fold excess (relative to the amount of rhGH in a measured aliquot of the stock rhGH) of the warmed mPEG-ButryALD was dissolved in 10 mM sodium phosphate (pH 7.2) to form a 10% reagent solution. The 10% reagent solution was quickly added to the aliquot of stock rhGH solution and mixed well. After the addition of the mPEG-ButryALD, the pH of the reaction mixture was determined and adjusted to 7.2 to 7.3 using conventional techniques, followed by mixing for thirty minutes. A reducing agent, sodium cyanoborohydride (NaCNBH₃), was then added at sixty to seventy molar excess relative to the rhGH (with the pH tested and adjusted using conventional techniques to ensure a pH of about 7.2 to 7.3). The reaction solution was thereafter stirred for five hours at room temperature in the dark. Then, the reaction solution was stirred overnight at 3-8° C. in a cold room in the dark to ensure coupling via a secondary amine linkage to thereby form a conjugate solution. The reaction was quenched with Tris buffer. The conjugate solution was characterized.

Lane 3 in FIG. 1 shows the SDS-PAGE analysis of this conjugate solution.

The aldehyde group of mPEG-ButyrALD can react with the primary amines associated with rhGH and covalently bond to them via secondary amine upon reduction by a reducing reagent such as sodium cyanoborohydride. This is because the PEGylation reaction was carried at pH 7.2 to 7.3, which favors internal lysine modification in conjunction with N-terminal modification. FIG. 4 shows the SEC-HPLC chromatogram of the conjugate solution. The PEGylation reaction yielded 53% 1-mer (one PEG attached to rhGH or monoPEGylated), mono(mPEG-ButyrALD-30 k) hGH, and 38% 2-mer (two PEGs attached to rhGH or diPEGylation) species, di(mPEG-ButyrALD-30 k) hGH.

An anion-exchange chromatography method using Q Sepharose Fast Flow and TRIS buffer was also used to purify the conjugates. The profile of the purified conjugates was similar to that shown in FIG. 2B. The purified conjugates were up to 100% pure.

Using this same approach, other conjugates can be prepared using mPEG-BuryrALD having other weight average molecular weights.

### Example 4

### PEGylation of rhGH with Branched mPEG-Butyraldehyde Derivative, 40 kDa

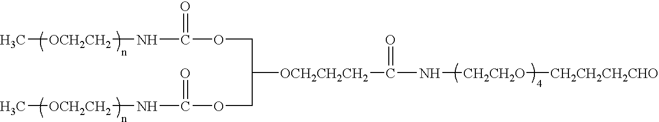

### Branched mPEG-Butyraldehyde Derivative, 40 kDa (“mPEG2-ButyrALD”)

mPEG-ButyrALD, 40 kDa, stored at −20° C. under argon, was warmed to ambient temperature. A ten-fold excess (relative to the amount of rhGH in a measured aliquot of the stock rhGH solution) of the warmed mPEG-ButryALD was dissolved in 10 mM sodium phosphate (pH 7.2) to form a 10% reagent solution. The 10% reagent solution was quickly added to the stock rhGH solution and mixed well. After the addition of the mPEG2-ButryALD, the pH of the reaction mixture was determined and adjusted to 7.2 to 7.3 using conventional techniques, followed by mixing for thirty minutes. A reducing agent, sodium cyanoborohydride (NaCNBH₃), was added at about seventy molar excess relative to the rhGH (with the pH tested and adjusted using conventional techniques to ensure a pH of about 7.2 to 7.3). To allow for coupling of the mPEG2-ButryALD to rhGH via a secondary amine linkage, the reaction solution was stirred for five hours at room temperature in the dark and thereafter stirred overnight at 3-8° C. in a cold room in the dark, thereby resulting in a conjugate solution. The reaction was quenched with Tris buffer. The conjugate solution was characterized.

Lane 1 in FIG. 1 shows the SDS-PAGE analysis of this conjugate solution.

The aldehyde group of mPEG2-ButyrALD can react with the primary amines associated with rhGH and covalently bond to them via a secondary amine upon reduction by a reducing reagent such as sodium cyanoborohydride. This is because the PEGylation reaction was carried at pH 7.2 to 7.3, which favors internal lysine modification in conjunction with N-terminal modification. FIG. 5 shows the SEC-HPLC chromatogram of the conjugate solution. The PEGylation reaction yielded 63% I-mer (one PEG attached to rhGH or monoPEGylated), mono(mPEG-2-ButyrALD-40 k) hGH, and 32% 2-mer (two PEGs attached to rhGH or diPEGylated), di(mPEG-2-ButyrALD-40 k) hGH. An anion-exchange chromatography method using Q Sepharose Fast Flow and Tris buffer was also developed to purify the conjugates. The profile of the purified conjugates was similar to that shown in FIG. 2B. The purified conjugates were up to 100% pure.

Using this same approach, other conjugates can be prepared using mPEG2-BuryrALD having other weight average molecular weights.

### Example 5

### PEGylation of hGH with mPEG-PIP, 20 kDa

mPEG-PIP having a molecular weight of 20,000 Daltons was obtained from Nektar Therapeutics (Huntsville, Ala.). The basic structure of the polymeric reagent is provided below:

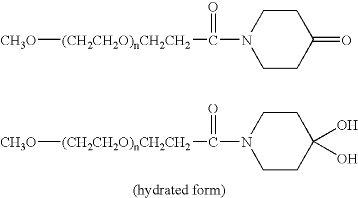

mPEG-PIP, 20 kDa, stored at −20° C. under argon, was warmed to ambient temperature. A twentyfive-fold excess (relative to the amount of rhGH in a measured aliquot of the stock rhGH) of the warmed mPEG-PIP was dissolved in 10 mM sodium phosphate (pH 7.2) to form a 10% reagent solution. The 10% reagent solution was quickly added to the aliquot of stock rhGH solution and mixed well. After the addition of the mPEG-PIP, the pH of the reaction mixture was determined and adjusted to 7.2 to 7.3 using conventional techniques, followed by mixing for thirty minutes. A reducing agent, sodium cyanoborohydride (NaCNBH₃), was then added at 125 molar excess relative to the rhGH (with the pH tested and adjusted using conventional techniques to ensure a pH of about 7.2 to 7.3). The reaction solution was thereafter stirred for five hours at room temperature in the dark. Then, the reaction solution was stirred for 28 hours at room temperature in the dark to ensure coupling via a secondary amine linkage (to a secondary carbon) to thereby form a conjugate solution. The reaction was quenched with Tris buffer.

The ketone group of mPEG-PIP can react with the primary amines associated with rhGH and covalently bond to them via secondary amine upon reduction by a reducing reagent such as sodium cyanoborohydride. The PEGylation reaction yielded about 30% 1-mer (one PEG attached to rhGH or monoPEGylated) and very little of di- or multi-PEGylated species.

Using this same approach, other conjugates can be prepared using mPEG-PIP having other weight average molecular weights.

### Example 6

### PEGylation of Engineered hGH with mPEG-MAL, 20 kDa

mPEG-Maleimide having a molecular weight of 20,000 Daltons is obtained from Nektar Therapeutics, (Huntsville, Ala.). The basic structure of the polymeric reagent is provided below:

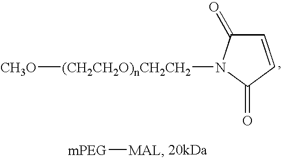

rhGH engineered to include a cysteine residue is dissolved in buffer. To this protein solution is added a 3-5 fold molar excess of mPEG-MAL, 20 kDa. The mixture is stirred at room temperature under an inert atmosphere for several hours. Analysis of the reaction mixture reveals successful conjugation of rhGH.

Using this same approach, other conjugates can be prepared using mPEG-MAL having other weight average molecular weights.

### Example 7

### In Vitro Activity of Exemplary (rhGH)-PEG Conjugates

The in vitro activities of the (rhGH)-PEG conjugates described in the preceding Examples are determined using one or more standard assays for assessing biological activity in vitro. Standard assays that may be employed include cell proliferation assays using, e.g., FDC-P1 cells (see, e.g., Clark et al., Journal of Biological Chemistry, 271:21969-21977, 1996), or Ba/F3-hGHR cells, which express receptors for hGH, hGH delta 135-146, or Nb2 rat lymphoma cells, which proliferate in response to hGH via the lactogenic receptors (see, e.g., Alam, K. S., et al., J. Biotech 2000, Feb. 28, 78(1), 49-59). Receptor binding assays (see, e.g., U.S. Pat. No. 5,057,417), and hypophysectomized rat growth (Clark, et al., Journal of Biological Chemistry, ibid) may also be used.

All of the rhGH conjugates described above are believed to be bioactive.

### Example 8

### Additional Characterization of Exemplary (rhGH)-PEG Conjugates

Additional physiochemical analyses were conducted on the conjugates described in Examples 1-6.

The table below provides a cross-reference for each of the conjugates analyzed and the corresponding Example in which its preparation and purification is described.

TABLE 4
Cross-Reference Information for Exemplary hGH Conjugates

| | Sample | Example Cross | |
|---|---|---|---|
| Conjugate Designation | Abbreviation | Reference | Description |
| mono(mPEG-2-NHS-40k) hGH | 1M | Example 1 | purified monomer |
| di(mPEG-2-NHS-40k) hGH | 1D | Example 1 | purified dimer |
| mono(mPEG-SMB-30k) hGH | 2M | Example 2 | purified monomer |
| di(mPEG-SMB-30k) hGH | 2D | Example 2 | purified dimer |
| mono(mPEG-ButyrALD-30k) hGH | 3M | Example 3 | purified monomer |
| di(mPEG-ButyrALD-30k) hGH | 3D | Example 3 | purified dimer |
| mono(mPEG-2-ButyrALD-40k) hGH | 4M | Example 4 | purified monomer |
| di(mPEG-2-ButyrALD-40k) hGH | 4D | Example 4 | purified dimer |

A. Protein Content.

The rhGH content of PEGylated and control samples was determined by several methods including the Bradford method (Bradford, M M. Analytical Biochemistry 72: 248-254. 1976), UV analysis at a wavelength of 280 nm in microtiter-plate format (5-point calibration), and by size exclusion chromatography with subsequent WV detection (280, 214 nm). The results confirmed covalent attachment of PEG; protein content for all samples was found to be in good agreement across the various methods employed.

B. SDS-PAGE Analysis

SDS-PAGE analysis, conducted under reducing conditions using both PEG stain and silver stain, was conducted on the purified conjugates described in Table 4 above. The observations based on the SDS-PAGE results were as follows:

For samples 1M and 1D: the analysis demonstrated that rhGH was successfully PEGylated. The purified monomer, 1M, contained a small amount of dimer, while the purified dimer, 1D, contained a small amount of monomer and higher molecular weight PEGylated species (high-mers).

For samples 2M and 2D: the analysis demonstrated that rhGH was successfully PEGylated. The purified monomer, 2M, contains a low amount of dimer, traces of higher PEGylated product and free rhGH, and a smear of low molecular weight conjugates. The purified dimer, 2D, contained a low amount of monomer, a considerable amount of tri-PEGylated product, and traces of free rhGH.

For samples 3M and 3D: the analysis demonstrated that rhGH was successfully PEGylated. The purified monomer, 3M, contained a low amount of dimer. The purified dimer, 3D, contained a low amount of monomer, a considerable amount of trimer, and traces of higher molecular weight conjugates (highmers).

For samples 4M and 4D: the analysis demonstrated that rhGH was successfully PEGylated. The purified monomer, 4M, contained a small amount of dimer. The purified dimer, 4D, contained a small amount of monomer and trimer, and traces of higher molecular weight PEGylated products (highmers).

C. Size Exclusion Chromatography

Size exclusion chromatography of the PEG conjugates provides information on purity, size distribution, and content. Although baseline separation of the main component and reaction by-products (mono-, di-, tri-PEGylated species, etc.) was not achieved under the conditions employed, qualitative information regarding size distribution in relation to unmodified hGH and estimates regarding purity were obtained.

For samples 1M and 1D: The purified monomer, 1M, showed a well-separated by-product of higher molecular weight, interpreted to be dimer. The purified dimer, 1D, exhibited a significant tailing, which most likely reflects the presence of monomer and/or a broader molecular weight distribution of the PEG-moiety.

For samples 2M and 2D: The purified monomer, 2M, showed a well-separated by-product of higher molecular weight, interpreted as the dimer. The purified dimer, 2D showed a moderate separated by-product of lower molecular weight, interpreted as the monomer.

For samples 3M and 3D: The purified monomer, 3M, showed a well-separated by-product of higher molecular weight, interpreted as the dimer. The purified dimer, 3D, showed a moderate separated by-product of lower molecular weight, interpreted as the monomer.

For samples 4M and 4D: The purified monomer, 4M, showed a well-separated by-product of higher molecular weight, interpreted as the dimer. The purified dimer, 4D, showed a moderately separated by-product of lower molecular weight, interpreted as the monomer.

Generally, the purity of the purified conjugate samples appeared higher in the monomer samples than in the dimer samples. A summary of calculated purity assessments based on SEC and UV-detection (214 nm) is provided in Table 5.

TABLE 5
Purity of Various hGH Conjugates

| Sample Description | Calculated Purity, % | Number of Impurities, >0.1% |
|---|---|---|
| 1M | 94.1 | 4 |
| 1D | 99.5 | 2 |
| 2M | 89.5 | 4 |
| 2D | 89.1 | 3 |
| 3M | 96.4 | 2 |
| 3D | 89.3 | 3 |
| 4M | 94.5 | 2 |
| 4D | 84.3 | 2 |

In general, with the exception of sample 4D, the purity of of the conjugates in all cases was about 90% or higher, and in some cases, was about 95% or higher, with the respect to type of conjugate (e.g., monoPEGylated—i.e., having only one PEG moiety attached to rhGH, diPEGylated—having two PEG moieties attached to rhGH, etc.).

### Example 9

### Biological Evaluations of Exemplary (rhGH)-PEG Conjugates

A. In Vitro Assay

The biological activity of rhGH and the conjugates described in Table 4 was assessed in vitro using an NB2-11 rat lymphoma cell proliferation assay. Briefly, NB2-11 cells derived from a rat lymphoma were incubated with rhGH, which leads to binding of the rhGH molecule to its receptor on the cell surface. Receptor binding induces the signal transduction cascade, which results in proliferation of the cells. Assay results were based on determined protein content, and a 100% bioactivity of unmodified rhGH. A summary of results is provided in Table 6.

TABLE 6
In vitro Activity of Exemplary hGH Conjugates

| Sample Description | Activity, % | Number of Assays |
|---|---|---|
| 1M | 3.6 | 2 |
| 1D | 0.4 | 1 |
| 2M | 15.5 | 2 |
| 2D | 2.7 | 2 |
| 3M | 11.4 | 2 |
| 3D | 1.4 | 2 |
| 4M | 2.5 | 2 |
| 4D | 0.2 | 1 |

In general, these results suggest that the dimers (di-PEGylated species) exhibited an approximate 7-fold decrease in in vitro activity compared to the corresponding monomers, while conjugates prepared using higher molecular weight PEGs (e.g., 40 kD versus 30 kD) exhibited an approximate 5-fold reduction in in vitro activity relative to conjugates prepared using the lower molecular weight PEGs.

B. In Vivo Assay

Conjugates 1M and 1D were further evaluated in a preliminary in vivo bioassay (hypophysectomized rat, USP monograph for Somatotropin®). Six groups of each of five animals received either rhGH, 1M, 1D, or placebo in different dosing regimes. Weight gain as a pharmacodynamic output was measured on day 1, 3, 6, and 8. Additional details are provided in Table 7 below.

TABLE 7
In vivo Study Details

| | | | | Cumulative |
|---|---|---|---|---|
| Study Group | Sample | Application | Dose | Dose |
| Std daily | rhGH Standard | daily | 5 μg | 35 μg |
| Placebo | placebo | daily | 0 μg | 0 μg |
| daily | | | | |
| Std bolus | rhGH Standard | single dose day 1 | 110 μg | 110 μg |
| Placebo | placebo | single dose day 1 | 0 μg | 0 μg |
| bolus | | | | |
| 1M bolus | 1M | single dose day 1 | 110 μg | 110 μg |
| 1D bolus | 1D | single dose day 1 | 110 μg | 110 μg |

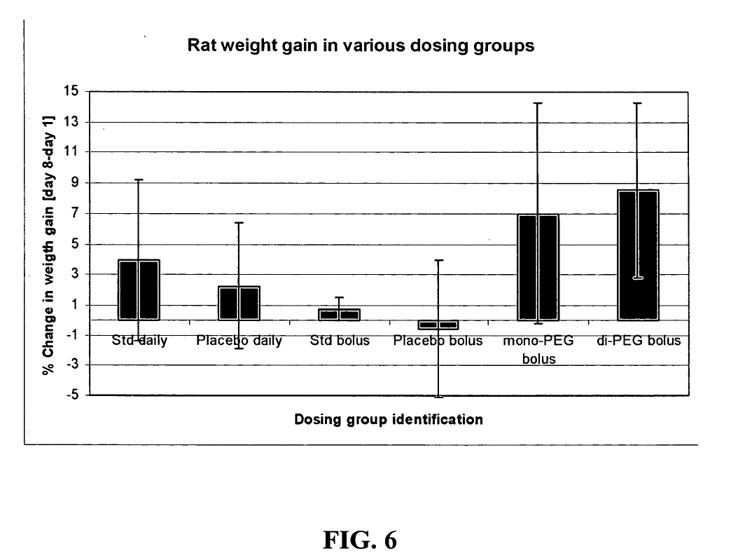

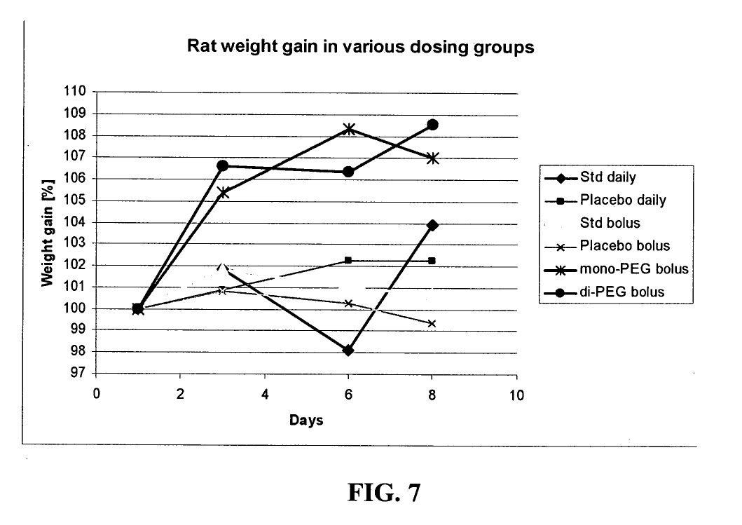

The results of the experiment are summarized in FIG. 6 (weight gain following day 8) and FIG. 7 (weight gain over time course of study). Data in FIG. 6 are given as mean±standard deviation, where n=5, except for the di-PEGylated sample, 1D, where n=4, due to the death of one animal). Placebo and a single high dose of rhGH resulted in no or moderate weight gain during the eight days. Daily dosing of 5 μg of the rhGH standard induced an overall weight gain of 4% on day 8, whereas the single high dose of 110 μg of 1M and 1D produced an increase of weight of 7% and 9% respectively (FIG. 6). Comparing the time course of weight gain, it appears that the PEGylated rhGH conjugates evoke a similar but superior response in the hypophysectomized rat (FIG. 7).

Although the preliminary in vitro results suggest that increasing the amount of PEG attached to hGH reduces its ability to stimulate the hGH receptor, based on the preliminary in vivo results, it appears that a reduction in bioactivity is more than balanced by increased half-life and/or plasma availability, thus leading to a conclusion that the conjugates provided herein possess a superior pharmacodynamic effect in vivo when compared to unmodified rhGH at an identical dosing regimen.

What is claimed is:

1. A conjugate comprising the following structure: 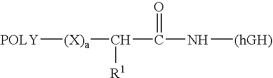
wherein POLY is a linear water-soluble polymer; (a) is either zero or one; X, when present, is a spacer moiety comprised of one or more atoms; R¹ is an organic radical containing 1 to 3 carbon atoms; and hGH is a residue of a human growth hormone (“hGH”) moiety.

2. The conjugate of claim 1, wherein the water-soluble polymer is a poly(ethylene glycol).

3. The conjugate of claim 2, wherein the poly(ethylene glycol) is terminally capped with methoxy.

4. The conjugate of claim 3, wherein the poly(ethylene glycol) has a weight-average molecular weight in the range of from about 6,000 Daltons to about 100,000 Daltons.

5. The conjugate of claim 4, wherein the poly(ethylene glycol) has a weight-average molecular weight in the range of from about 10,000 Daltons to about 85,000 Daltons.

6. The conjugate of claim 5, wherein the poly(ethylene glycol) has a weight-average molecular weight in the range of from about 20,000 Daltons to about 65,000 Daltons.

7. The conjugate of claim 4, wherein the hGH moiety is human growth hormone or a biologically active fragment, deletion variant, substitution variant or addition variant thereof.

8. The conjugate of claim 7, wherein the hGH moiety is human growth hormone.

9. The conjugate of claim 7, wherein the hGH moiety comprises the amino acid sequence of SEQ ID NO: 1.

10. The conjugate of claim 1, comprising the following structure: 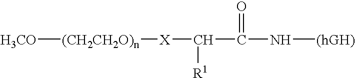
wherein:
(n) is an integer having a value of from 150 to 2200;
X is an acyclic spacer moiety comprising from 1 to about 25 atoms;
R¹ is an organic radical containing 1 to 3 carbon atoms selected from the group consisting of methyl, ethyl, propyl, and isopropyl; and
hGH is a residue of human growth hormone.

11. The conjugate of claim 10, wherein X is an alkylene chain comprising from 1 to 10 carbon atoms.

12. The conjugate of claim 11, comprising the following structure: 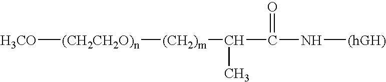
wherein (m) is an integer ranging from 1 to 8.

13. The conjugate of claim 12, comprising the following structure: 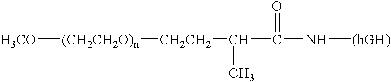
wherein (n) an integer having a value of from 150 to 2200.

14. The conjugate of claim 13, in monoPEGylated form.

15. The conjugate of claim 13, in diPEGylated form.

16. A composition comprising: 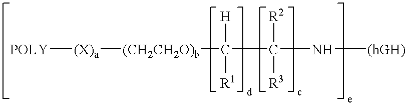
wherein:
POLY is a water-soluble polymer;
(a) is either zero or one;
(b) is either 0 or an integer having a value from 1 to 10;
(c) is an integer having a value from 1 to 10;
(d) is either zero or one;
(e) is either one or two;
X, when present, is a spacer moiety comprised of one or more atoms;
R¹ is H or an organic radical containing from 1 to 3 carbon atoms;
Either R² and R³, when taken together with the carbon atom to which they are attached, in each occurrence considered separately, forms a carbonyl (C═O), or
R², in each occurrence, is independently H or an organic radical, and
R³, in each occurrence, is independently H or an organic radical; and
hGH is a residue of a human growth hormone residue,
wherein the following provisos apply:
(i) when R² and R³, when taken together with the carbon atom to which they are attached form a carbonyl adjacent to the —NH—, and POLY is linear, then (d) equals one and R¹ is an organic radical containing from 1 to 3 carbon atoms;
(ii) when R² and R³, in each occurrence, are each independently H or an organic radical, then (b) is a positive integer and (a) is one;
(iii) when POLY is branched, POLY does not contain a lysine residue,
and further wherein when (e) is one, the composition comprises 85% or greater monoconjugate with respect to all conjugate species contained in the composition, and when (e) is two, the composition comprises greater than 80% diconjugate with respect to all conjugate species contained in the composition, and said composition is bioactive.

17. The composition of claim 16, wherein the water-soluble polymer is a poly(ethylene glycol).

18. The composition of claim 17, wherein the poly(ethylene glycol) is terminally capped with methoxy.

19. The composition of claim 18, wherein the poly(ethylene glycol) has a weight-average molecular weight in the range of from about 6,000 Daltons to about 100,000 Daltons.

20. The composition of claim 19, wherein the poly(ethylene glycol) has a weight-average molecular weight in the range of from about 10,000 Daltons to about 85,000 Daltons.

21. The composition of claim 20, wherein the poly(ethylene glycol) has a weight-average molecular weight in the range of from about 20,000 Daltons to about 65,000 Daltons.

22. The composition of claim 16, wherein the hGH moiety is human growth hormone or a biologically active fragment, deletion variant, substitution variant or addition variant thereof.

23. The composition of claim 22, wherein the hGH moiety is human growth hormone.

24. The composition of claim 23, wherein hGH moiety comprises the amino acid sequence of SEQ ID NO:1.

25. The composition of claim 16, wherein (e) is one.

26. The composition of claim 16, wherein (e) is two.

27. The composition of claim 16, wherein X is an acyclic spacer moiety comprising from 1 to about 25 atoms.

28. The composition of claim 18, wherein R² and R³, when taken together with the carbon atom to which they are attached, form a carbonyl adjacent to the —NH—, and POLY is branched.

29. The composition of claim 28, wherein (b) and (d) both equal zero, and (c) equals 4.

30. The composition of claim 29, comprising:

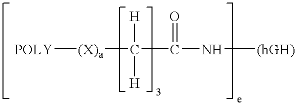

31. The composition of claim 30, where (a) is zero.

32. The composition of claim 31, wherein the branched water-soluble polymer comprises the following structure: 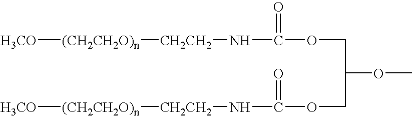
wherein each (n) is independently an integer having a value of from 150 to 2200.

33. The composition of claim 16, where POLY is branched, (a) is one, (b) ranges from 1 to 10, (c) ranges from 1 to 6, and (d) is zero.

34. The composition of claim 33, where (b) is 4.

35. The composition of claim 34, where (c) is 4 and R² and R³ are both H in each occurrence.

36. The composition of claim 35, where X comprises —(CH₂)₃—C(O)—NH—.

37. The composition of claim 36, where POLY comprises the following structure: 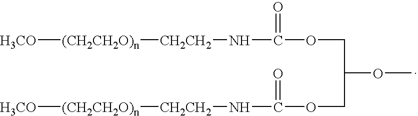
wherein each (n) is independently an integer having a value of from 150 to 2200.

38. The composition of claim 18, wherein POLY is linear, (a) is one, (b) ranges from 1 to 10, (c) ranges from 1-6, and (d) is zero.

39. The composition of claim 38, wherein (b) is 4.

40. The composition of claim 39, where (c) is 4 and R² and R³ are both H in each occurrence.

41. The composition of claim 40, where X comprises —C(O)—NH—.

42. The composition of claim 41, wherein POLY is H₃CO—(CH₂CH₂O)ₙ— and (n) is an integer having a value of from 150 to 2200.

43. The composition of claim 16 further comprising a pharmaceutical excipient.

44. The composition of claim 16, wherein the composition is substantially free of albumin.

45. The composition of claim 44, wherein the composition is substantially free of proteins that do not possess hGH activity.

46. The composition of claim 45, wherein the composition is substantially free of noncovalently attached water-soluble polymers.

47. The composition of claim 46, in lyophilized form.

48. The composition of claim 46, in the form of a liquid.

49. The composition of claim 16, wherein the amount of monoconjugate and diconjugate in the composition represents at least about 85% of the total conjugate species in the composition.

50. The composition of claim 49, wherein the amount of monoconjugate and diconjugate in the composition represents at least about 90% of the total conjugate species in the composition.

51. The composition of claim 50, wherein the amount of monoconjugate and diconjugate in the composition represents at least about 95% of the total conjugate species in the composition.


SEQUENCE LISTING (4 sequences)

SEQ ID NO: 1 - protein, 191 aa, Homo sapiens [sequence omitted]

SEQ ID NO: 2 - protein, 192 aa, Homo sapiens
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 3 - protein, 191 aa, Homo sapiens
FPTIPLSRLFDNAMLRAHRLHQLAFDTYQEFEEAYIPKEQKYSFLQNPQTSLCFSESIPTPSNREETQQKSNLELLRISLLLIQSWLEPVQSLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 4 - protein, 192 aa, Homo sapiens
MFPTIPLSRLFDNAMLRAHRLHQLAFDTYQEFEEAYIPKEQKYSFLQNPQTSLCFSESIPTPSNREETQQKSNLELLRISLLLIQSWLEPVQSLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF